(19) United States
(12) Patent Application Publication
(10) Pub. No.: US 2007/0149600 A1
(43) Pub. Date: Jun. 28, 2007

(54) Substituted octahydroisoindoles

(76) Inventors: Andrew Browning, Knivsta (SE); Martin Scobie, Uppsala (SE)

Correspondence Address: FISH & RICHARDSON PC, P.O. BOX 1022, MINNEAPOLIS, MN, 55440-1022, US

(21) Appl. No.: 11/409,704

(22) Filed: Apr. 24, 2006

Related U.S. Application Data
(60) Provisional application No. 60/677,149, filed on May 3, 2005.

(30) Foreign Application Priority Data
Apr. 25, 2005 (SE) 0500918-8

(51) Int. Cl. A61K 31/4035; C07D 405/02; C07D 209/44

(52) U.S. Cl. 514/414; 514/416; 548/454; 548/470

(57) ABSTRACT

The invention relates to compounds of the general formula (I) 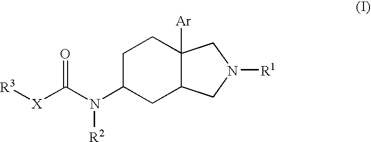
wherein R¹, R², R³, Ar, and X are as defined herein, or a pharmaceutically acceptable salt, hydrates, geometrical isomers, racemates, tautomers, optical isomers, N-oxides and prodrug forms thereof. The compounds may be used for the treatment or prophylaxis of disorders related to the MCH1R receptor and for modulation of appetite. The invention also relates to such use as well as to pharmaceutical formulations comprising a compound of formula (I).

### RELATED APPLICATION INFORMATION

This application claims priority from U.S. Provisional Application No. 60/677,149, filed May 3, 2005, and Swedish Application No. 0500918-8, filed Apr. 25, 2005, both of which are hereby incorporated by reference in their entirety.

### TECHNICAL FIELD

The present invention relates to substituted octahydroisoindoles that act as antagonists of melanin concentrating hormone receptor 1 (MCH1R). The invention further relates to pharmaceutical compositions comprising these compounds, and to the use of the compounds for the preparation of a medicament for the prophylaxis or treatment of obesity and other disorders as well as method of treatment of these disorders.

### BACKGROUND ART

Melanin Concentrating Hormone (MCH) is a 19 amino acid cyclic peptide, which is expressed in hypothalamus in the mammalian brain (Nahon J L et al., Endocrinology, 1989; 125(4):2056-65 and Tritos N A, et al., Diabetes, 1998; 47(11): 1687-92). A substantial body of evidence has shown that this peptide plays a critical role in the moderation of feeding behaviour and energy expenditure. Studies have shown that ICV administration of MCH directly into rat brains results in a marked increase in food intake (Ludwig D S et al., Am. J. Physiol., 1998; 274(4 Pt 1):E627-33). It has also been shown that messenger RNA for the MCH precursor is up-regulated in the hypothalamus of fasted animals and in animals that are genetically obese (Qu D, Ludwig D S et al., Nature, 1996; 380(6571):243-7). Furthermore, mice lacking MCH are hypophagic and lean, and have increased energy expenditure (20% increase over control animals when expressed on a per kg basis) (Shimada M et al., Nature, 1998; 396(6712):670-4). Studies of transgenic mice overexpressing MCH in the lateral hypothalamus show that these animals are more prone to diet-induced obesity when fed a high fat diet, and they have higher systemic leptin levels (Ludwig D S et al., J. Clin. Invest., 2001; 107(3):379-86). Blood glucose levels were increased both preprandially and after intraperitoneal injection of glucose. The animals also had increased insulin levels and insulin tolerance test indicated peripheral insulin resistance. Further support for the role of MCH in metabolic regulation comes from studies showing that, in mice, mRNA for the MCH receptor is upregulated 7-fold by 48 h fasting and in genetic leptin deficiency (ob/ob mice). These effects could be completely blunted by leptin treatment (Kokkotou E G et al., Endocrinology, 2001; 142(2):680-6.). In addition to its role in regulating feeding behavior, MCH antagonists have been demonstrated to have anxiolytic and antidepressant effects (Borowsky, B D et al., Nature Medicine, 2002. 8(8): 825-830).

Obesity is linked to a wide range of medical complications, such as diabetes, cardiovascular disease and cancer. In addition, being overweight can exacerbate the development of osteoporosis and asthma. For example, at least 75% of Type II diabetics are overweight and a clear correlation has been demonstrated between weight and the prevalence of Type II diabetes. Obesity is also proven to double the risk of hypertension. It is estimated that between 2% and 8% of total health-care costs in the Western world are related to obesity, i.e. in excess of 10 billion USD.

Initial treatment for obesity is simple diet and exercise. Initial drug therapy tends to be focused around suppression of appetite. Many of the older appetite-suppressant agents act via the noradrenergic (and possibly dopaminergic) receptors to produce a feeling of satiety. Amphetamine was the archetypal agent in this class, but it has substantial potential for stimulating the central nervous system and consequent abuse. More recent developments, such as Xenical (orlistat), marketed by Roche, have focused on preventing fat absorption in the gut. Xenical inhibits the action of the enzyme lipases, thereby reducing the digestion of triglycerides and subsequent absorption by the intestinal tract. Unfortunately, this does not address overeating and excess calorie intake. Other pharmacological approaches for the treatment of obesity include serotonin re-uptake inhibitors, such as Reductil (sibutramine) marketed by Abbot, which acts as an appetite-suppressant.

The concept of using MCH1R antagonists for the treatment of obesity has recently been published (Carpenter and Hertzog, Expert Opin. Ther. Patents, 2002, 12(11): 1639-1646, Collins and Kym, Curr. Opin. In Invest. Drugs, 2003, 4(4): 386-394, Browning, Expert Opin. Ther. Patents, 2004, 14(3): 313-325 and Kowalski and McBriar, Expert Opin. Investig. Drugs, 2004, 13(9): 1113-1122).

WO01/21169 (Takeda Chemical Industries) describes diaryl compounds as MCH-1R antagonists useful for the treatment of obesity. JP13226269 (Takeda), describing several piperidine-substituted benzazepines and benzazepinones; WO01/82925 (Takeda), disclosing different amines; and WO01/87834 (Takeda) describing piperidine compound with benzene (1:1), claim compounds for the treatment of obesity. WO01/21577 (Takeda) discloses a series of amines claimed to be anorectic, anti-diabetic and antidepressant agents. WO01/57070 (Merck) describes in a series of truncated and modified peptidic MCH analogues as either significant agonist or antagonist activity. In WO02/10146 (GlaxoSmithKline) the preparation of carboxamide compounds claimed for the treatment of obesity as well as diabetes, depression and anxiety is disclosed. WO02/04433 (The Neurogen Corporation) describes in N-arylpiperazine derivatives and related 4-arylpiperidine derivatives as selective modulators of MCH-1R for the treatment of a variety of metabolic, feeding and sexual disorders. In WO02/06245 (Synaptic Pharmaceutical Corporation) a class of dihydropyrimidinones as MCH-1R antagonists for the treatment of feeding disorders, such as obesity and bulimia is disclosed. In WO02/051809 (Schering Corporation) 4-substituted piperidine derivatives are disclosed as MCH antagonists as well as their use in the treatment of obesity. In WO02/057233 aryl-substituted ureas are disclosed as MCH antagonists as well as their use in the treatment of obesity. The central core in the WO02/057233 is a(n) (hetero)arylene group.

PCT/SE2004/001619 and PCT/SE2004/001620 disclose octahydroindoles that act as antagonists for the melanin concentrating hormone receptor 1 (MCH1R).

Mesembrine, 3a-(3,4-dimethoxyphenyl)-1-methyloctahydro-6H-indol-6-one, is a natural product obtained as an extract of plants of the Mesembryanthemaceae family, including Sceletium tortuosum. In small doses the mesembrine extracts have a meditative or narcotic effect. Hottentots used Sceletium expansum and tortuosum as a psychedelic called “channa”. The use of mesembrine as a serotonin-uptake inhibitor for the treatment of an array of mental disorders is disclosed in WO97/46234.

U.S. Pat. No. 6,288,104 discloses mesembrine-like compounds lacking the urea or amide group in the present compounds. This document relates to serotonin-uptake inhibitors for the treatment of depression and anxiety.

### DISCLOSURE OF THE INVENTION

According to the present invention, novel substituted octahydroisoindoles have been found that are active towards the MCH1R receptor. The compounds are relatively easy to prepare and can be used for the treatment or prevention of obesity, diabetes mellitus, hyperlipidemia, hyperglycemia, modulation of appetite, depression, anxiety or urinary incontinence. The compounds can further be used in conjunction with other compounds acting through other mechanisms, such as MC-4 agonists, 5HT_{2c} agonists, or 5HT₆ antagonists. The compounds can also be used in conjunction with anti-obesity medicaments.

Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted by one or more halogen atoms.

Unless otherwise stated or indicated, the term “C₁₋₆-alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc. “Halo-C₁₋₆-alkoxy” means a C₁₋₆-alkoxy group substituted by one or more halogen atoms.

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring for example, a C₆ ring or a C₁₀ fused ring system.

Examples of aryls are phenyl, pentalenyl, indenyl, dihydroindenyl, isoindolinyl, chromanyl, naphthyl, fluorenyl, anthryl, phenanthryl and pyrenyl. The aryl rings may optionally be substituted by C₁₋₆-alkyl. Examples of substituted aryl groups are benzyl and 2-methylphenyl.

The term “heteroaryl” refers to a hydrocarbon ring system having at least one aromatic ring which contains at least one heteroatom such as O, N, or S. Examples of heteroaryl groups include furyl, pyrrolyl, thienyl, oxazolyl, isoxazolyl, imidazolyl, thiazolyl, isothiazolyl, pyridyl, pyrimidinyl, quinazolinyl, indolyl, pyrazolyl, pyridazinyl, quinolinyl, quinoxalinyl, thiadiazolyl, benzofuranyl, 2,3-dihydrobenzofuranyl, benzodioxolyl, benzodioxinyl, 2,3-dihydro-1,4-benzodioxinyl, benzothiazolyl, benzothiadiazolyl, and benzotriazolyl groups.

The term “coupling agent” refers to a compound used when coupling together an amine and a carboxylic acid. An example of a coupling agent is 1-[3-(dimethylaminopropyl)]-3-ethylcarbodiimide hydrochloride, which is used in the presence of hydroxybenzotriazole and a base such as triethylamine.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

When two of the above-mentioned terms are used together, it is intended that the latter group is substituted by the former. For example, halo-C₁₋₆-alkyl means a C₁₋₆-alkyl group that is substituted by one or more halogen atoms. Likewise, halo-C₁₋₆-alkylthio means a C₁₋₆-alkylthio group that is substituted by one or more halogen atoms.

The following abbreviations have been used:

DCM means dichloromethane,

DMF means dimethylformamide,

i-PrOH means isopropanol,

HPLC means high performance liquid chromatography,

HRMS means high resolution mass spectrometry,

R.T. (rt.) means room temperature,

TFA means trifluoroacetic acid,

THF means tetrahydrofuran.

In a first aspect, the present invention provides a compound of the general formula (I) 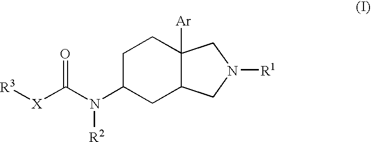
or a pharmaceutically acceptable salt, hydrates, geometrical isomers, racemates, tautomers, optical isomers, N-oxides and prodrug forms thereof, wherein:
X is NH or a single bond;
Ar is aryl, wherein the aryl may be substituted by one or more of C₁₋₆-alkoxy;
R¹ is C₁₋₆-alkyl;
R² is H;
R³ is selected from aryl optionally independently substituted by one or more of halogen, cyano, halo-C₁₋₆-alkylthio, C₁₋₆-alkoxy, C₁₋₆-alkyl, halo-C₁₋₆-alkyl; and heteroaryl optionally independently substituted by one or more halogen.

It is preferred that Ar is selected from phenyl, wherein the phenyl may be optionally independently substituted by one or more of C₁₋₆-alkoxy, especially by one or more of methoxy. Most preferably, Ar is 3,4-dimethoxyphenyl.

It is preferred that R¹ is methyl.

It is preferred that R³ is selected from phenyl optionally independently substituted by one or more of bromo, chloro, cyano, (difluoromethyl)thio, fluoro, iodo, methoxy, methyl, trifluoromethyl, (trifluoromethyl)thio; 1,3-benzodioxol-5-yl optionally substituted by one or more of fluoro; and pyridyl.

It is more preferred that R³ is selected from 4-bromo-3-methylphenyl, 3-bromophenyl, 4-bromophenyl, 4-bromo-2-(trifluoromethyl)phenyl, 3-chloro-2-fluorophenyl, 3-chloro-4-fluorophenyl, 3-chlorophenyl, 4-chlorophenyl, 4-chloro-3-(trifluoromethyl)phenyl, 3-cyanophenyl, 4-cyanophenyl, 2,4-dichlorophenyl, 2,5-dichlorophenyl, 3,4-dichlorophenyl, 2,2-difluoro-1,3-benzodioxol-5-yl, 3-(difluoromethyl)thiophenyl, 2,5-difluorophenyl, 3,4-difluorophenyl, 3,5-difluorophenyl, 3,4-dimethoxyphenyl, 3,5-dimethylphenyl, 3-fluorophenyl, 4-fluorophenyl, 2-fluoro-3-(trifluoromethyl)phenyl, 2-fluoro-4-(trifluoromethyl)phenyl, 3-fluoro-4-(trifluoromethyl)phenyl, 3-fluoro-5-(trifluoromethyl)phenyl, 4-fluoro-3-(trifluoromethyl)phenyl, 4-Iodophenyl, 2,3,4-trifluorophenyl, 3,4,5-trifluorophenyl, 3-(trifluoromethyl)phenyl, 4-(trifluoromethyl)phenyl, 4-(trifluoromethyl)thiophenyl, and 4-pyridyl.

Preferred compounds are denoted in Examples 7-29 and 31-56 below.

All diastereomeric forms possible (pure enantiomers, tautomers, racemic mixtures and unequal mixtures of two enantiomers) are within the scope of the invention. Such compounds can also occur as cis- or trans-, E- or Z-double bond isomer forms. All isomeric forms are contemplated.

Another object of the present invention is a process for the preparation of a compound above comprising at least one of the following steps:

(a) acylation of an amine with a substituted benzoyl chloride,

(b) treatment of an amine with 4-carboxypyridine-N-oxide in the presence of a coupling agent,

(c) treatment of an amine with a substituted aryl isocyanate,

(d) treatment of an amine with triphosgene and another amine.

Another object of the present invention is a compound as described above for use in therapy. The compound can be used in the treatment or prophylaxis of obesity, diabetes mellitus, hyperlipidemia, hyperglycemia, depression, anxiety, urinary incontinence, and for modulation of appetite. It may also be used in the treatment or prophylaxis of disorders relating to the MCH1R receptor and for modulation of appetite. Examples of such disorders are obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c} agonists, or 5HT₆ antagonists. The compound can also be used in conjunction with anti-obesity medicaments.

Another object of the present invention is a pharmaceutical formulation containing a compound as described above as an active ingredient, in combination with a pharmaceutically acceptable diluent or carrier. The pharmaceutical formulation may be used in the treatment or prophylaxis of obesity wherein the active ingredient is a compound as described above.

Another object of the present invention is a method for the treatment or prophylaxis of obesity, diabetes mellitus, hyperlipidemia, hyperglycemia, depression, anxiety, urinary incontinence, and for modulation of appetite, said method comprising administering to a subject (e.g., mammal, human, or animal) in need of such treatment an effective amount of a compound as described above. The compound can further be used in conjunction with other compounds active towards other receptors, such as MC-4 agonists, 5HT_{2c} agonists, or 5HT₆ antagonists. The compound can also be used in conjunction with anti-obesity medicaments.

Another object of the present invention is a method for the treatment or prophylaxis of disorders related to the MCH1R receptor and for modulation of appetite, said method comprising administering to a subject (e.g., mammal, human, or animal) in need of such treatment an effective amount of a compound as described above. The MCH1R receptor related disorder is any disorder or symptom wherein the MCH1R receptor is involved in the process or presentation of the disorder or the symptom. The MCH1R related disorders include, but are not limited to: obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c} agonists, or 5HT₆ antagonists. The compound can also be used in conjunction with anti-obesity medicaments.

Another object of the present invention is a method for modulating MCH1R receptor activity (e.g., antagonizing the human MCH1R receptor), comprising administering to a subject (e.g., mammal, human, or animal) in need thereof an effective amount of a compound as described above or a composition comprising a compound as described above.

Another object of the present invention is the use of a compound as described above in the manufacture of a medicament for use in the treatment or prophylaxis of obesity, diabetes mellitus, hyperlipidemia, hyperglycemia, depression, anxiety, and urinary incontinence, and for modulation of appetite.

Another object of the present invention is the use of a compound as described above in the manufacture of a medicament for use in the treatment or prophylaxis of disorders related to the MCH1R receptor and for modulation of appetite, said method comprising administering to a subject (e.g., mammal, human, or animal) in need of such treatment an effective amount of a compound as described above. The MCH1R receptor related disorder is any disorder or symptom wherein the MCH1R receptor is involved in the process or presentation of the disorder or the symptom. The MCH1R related disorders include, but are not limited to obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c} agonists, or 5HT₆ antagonists. The compound can also be used in conjunction with anti-obesity medicaments.

The compounds of the formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The necessary starting materials for preparing the compounds of formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following non-limiting Examples.

### COMPARATIVE EXAMPLE 1

### 3-(3,4-dimethoxyphenyl)-1-methyl-1H-pyrrole-2,5-dione

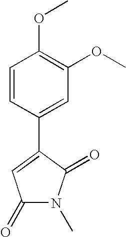

3,4-dimethoxyaniline (7.66 g, 50.0 mmol) was suspended in water/conc. HCl (2:3), (25 mL) with gentle heating. The reaction vessel was cooled (ice-bath) and some additional ice added to the reaction mixture. NaNO₂ in water (13 mL) was added dropwise to the thick suspension. The mixture was stirred at 0-5° C. for 20 min, a dark brown foam was formed, which was removed by filtration. The filtrate was poured into an ice-cold solution of N-methylmaleimide (8.08 g, 72.7 mmol) in acetone (40 mL). Solid NaOAc was added to the mixture to adjust the pH from 2 to 3. CuCl₂.2H₂O (1.31 g, 7.7 mmol) was added with continous stirring and the temperature maintained at 0-5° C. for 30 min. The temperature was then increased to 35° C. and stirring continued over night for 20 hours. The acetone was evaporated and the remaining dark viscous oil was extracted with toluene (4×120 mL). The organic layers were combined, dried and concentrated. The residue was partly dissolved in i-PrOH (30 mL) and 2,6-dimethylpyridine was added. The mixture was heated to 50° C. and after 20 min a brown precipitation was noticed. The mixture was stirred for 2.5 h, water (20 mL) was added, and the mixture was cooled in a fridge. The mixture was then filtered and the collected solid washed with i-PrOH and then ether to give the title compound 2.98 g, 24% as a gold-brown solid. HPLC purity 95%, 1H NMR (400 MHz, CHLOROFORM-D) δ ppm 3.04 (s, 3H) 3.92 (s, 6H) 6.58 (s, 1H) 6.90 (d, J=8.53 Hz, 1H) 7.48 (d, J=2.01 Hz, 1H) 7.60 (dd, J=8.53, 2.01 Hz, 1H).

### COMPARATIVE EXAMPLE 2

### (3aS*,7aR*)-7a-(3,4-dimethoxyphenyl)-2-methyltetrahydro-1H-isoindole-1,3,5(2H,4H)-trione

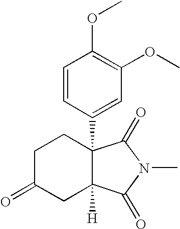

2-(Trimethylsilyloxy)-1,3-butadiene and 3-(3,4-dimethoxyphenyl)-1-methyl-1H-pyrrole-2,5-dione, prepared in comparative example 1, (6.947 g, 42.7 mmol) were dissolved in dry toluene (200 mL) and stirred at reflux for 64 h. The solvent was evaporated and the residue was dissolved in DCM (200 mL). Concentrated HCl was added dropwise with stirring over 25 min. The mixture was diluted with DCM and extracted with sat. aq. NaHCO₃. The layers were separated and the aqueous layer was extracted once with DCM. The organic layers were combined, dried and concentrated to give 9.87 g of a yellow solid of 75% purity. Purification by flash chromatography (1% MeOH in DCM) yielded 4.28 g (40%) of a yellow solid. HPLC purity 95%, 1H NMR (400 MHz, CHLOROFORM-D) δ ppm 1.98-2.09 (m, 1H) 2.30-2.44 (m, 3H) 2.55-2.63 (m, 1H) 2.83 (dd, J=16.82, 7.78 Hz, 1H) 2.93-3.00 (m, 1H) 3.06 (s, 3H) 3.53-3.57 (m, 1H) 3.86 (s, 3H) 3.88 (s, 3H) 6.84 (d, J=8.53 Hz, 1H) 6.88-6.92 (m, 1H). MS (ESI+) for C₁₇H₁₉NO₅ m/z 318 (M+H)⁺.

### COMPARATIVE EXAMPLE 3

### 7a′-(3,4-dimethoxyphenyl)-2′-methyltetrahydrospiro[1,3-dioxolane-2,5′-isoindole]-1′,3′(2′H,4′H)-dione E05760001

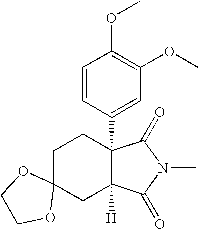

Glycol (570 μL, 10.2 mmol) and benzenesulfonic acid (25 mg, 0.17 mmol) were added to a solution of (3aS*,7aR*)-7a-(3,4-dimethoxyphenyl)-2-methyltetrahydro-1H-isoindole-1,3,5(2H,4H)-trione, prepared in comparative example 2. The solution was refluxed at 115° C. using a Dean-Stark trap for 3 hours. The reaction mixture was allowed to cool and was extracted with sat. aq. NaHCO₃ and brine. The organic layer was dried and concentrated to give 320 mg of the title compound as a yellow solid. HPLC purity 86%, MS (ESI+) for C₁₉H₂₃NO₆ m/z 362 (M+H)⁺.

### COMPARATIVE EXAMPLE 4

### (3aS*,7aR*)-7a-(3,4-dimethoxyphenyl)-2-methyloctahydrospiro[1,3-dioxolane-2,5-isoindole]

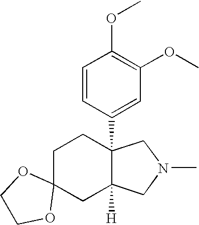

7a′-(3,4-dimethoxyphenyl)-2′-methyltetrahydrospiro[1,3-dioxolane-2,5′-isoindole]-1′,3′(2′H,4′H)-dione, prepared in comparative example 3, (3.41 g, 9.44 mmol) in THF (10 mL) was added by syringe to a cold suspension of LiAlH₄ (1.82 g, 47.9 mmol) in THF (60 mL). The mixture was heated to 65° C. and stirred for 2 hours. The vessel was cooled in an ice-bath and the reaction was quenched with water (6.39 mL). The resulting suspension was diluted with ether and filtered through celite which was rinsed several times with ether. The solvent was evaporated and the remaining residue was partitioned between DCM and 1M aq. NaOH. Drying and concentration of the organic layer gave 2.68 g (85%) of the title compound. HPLC purity 98%, MS (ESI+) for C₁₉H₂₇NO₄ m/z 334 (M+H)⁺.

### COMPARATIVE EXAMPLE 5

### (3aS*,7aR*)-7a-(3,4-dimethoxyphenyl)-2-methyloctahydro-5H-isoindol-5-one

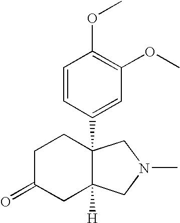

To a solution of (3aS*,7aR*)-7a-(3,4-dimethoxyphenyl)-2-methyloctahydrospiro[1,3-dioxolane-2,5-isoindole], prepared in comparative example 4, (2.68 g, 8.04 mmol) in THF (40 mL), 1M aq. HCl was added. The solution was stirred for 1.5 hours at 65° C. The reaction mixture was diluted with ether then extracted with 1M aq. NaOH. The layers were separated and the water phase was extracted once more with ether. The organic layers were combined, dried and concentrated to give 2.10 g (90%) of the title compound. HPLC purity 98%, 1H NMR (500 MHz, CHLOROFORM-D) δ ppm 2.08-2.14 (m, 1H) 2.14-2.20 (m, 1H) 2.30-2.37 (m, 1H) 2.33 (s, 3H) 2.41-2.48 (m, 1H) 2.55 (dd, J=14.89, 7.32 Hz, 1H) 2.59 (dd, J=9.28, 4.03 Hz, 1H) 2.63 (dd, J=15.01, 6.71 Hz, 1H) 2.68 (dd, J=7.69 Hz, 1H) 2.71 (d, J=9.16 Hz, 1H) 2.93 (ddd, J=14.01, 7.29, 4.21 Hz, 1H) 3.04 (d, J=9.16 Hz, 1H) 3.87 (s, 3H) 3.88 (s, 3H) 6.79-6.83 (m, 3H). MS (ESI+) for C₁₇H₂₃NO₃ m/z 290 (M+H)⁺. The relative stereochemistry of this structure was further confirmed by NOE experiment.

### COMPARATIVE EXAMPLE 6

### (3aS*,5R*,7aR*)-7a-(3,4-dimethoxyphenyl)-2-methyloctahydro-1H-isoindol-5-amine

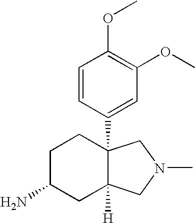

(3aS*,7aR*)-7a-(3,4-dimethoxyphenyl)-2-methyloctahydro-5H-isoindol-5-one, prepared in comparative example, 5 (100 mg, 0.31 mmol) was aliquoted into 6 reaction vials and MeOH (5 ml) which had been saturated with ammonia gas was added to each vial. Sodium cyanoborohydride (35 mg, 0.56 mmol) was added to each vial and the mixtures were irradiated in a Smith microwave apparatus at 120° C. for 400 seconds. HPLC analysis showed consumption of starting material and the presence of the expected products (1:1 cis/trans) in all the vials. The mixtures were combined and evaporated and the residues partitioned between 0.5N NaOH and chloroform. Evaporation of the organic phase gave a light yellow liquid which was purified by flash-chromatography over silica using chloroform saturated with ammonia gas as the eluent. This gave (3aS*,5R*,7aR*)-7a-(3,4-dimethoxyphenyl)-2-methyloctahydro-1H-isoindol-5-amine 0.172 g (30%) as a colorless oil. HPLC purity 99% MS (electrospray; [M+H]⁺) m/z: 291.1.

1H NMR (400 MHz, CHLOROFORM-D) δ ppm 0.90-1.03 (m, 1H) 1.07-1.34 (m, 2H) 1.35-1.47 (m, 1H) 1.55-1.65 (m, 1H) 1.80-1.89 (m, 1H) 1.95-2.10 (m, 2H) 2.38 (s, 3H) 2.62 (d, J=9.03 Hz, 1H) 2.73-2.81 (m, J=9.03, 9.03 Hz, 1H) 2.83-3.02 (m, 4H) 3.84 (s, 3H) 3.85 (s, 3H) 6.76-6.83 (m, 2H) 6.85-6.89 (m, 1H).

### EXAMPLE 7

### N-[(3aS*,5R*,7aR*)-7a-(3,4-dimethoxyphenl-2-methyloctahydro-1H-isoindol-5-yl]-4-iodobenzamide

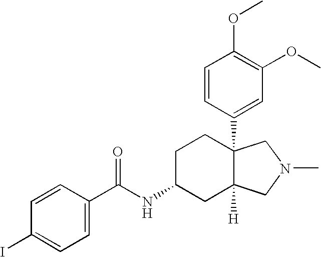

A solution of (3aS*,5R*,7aR*)-7a-(3,4-dimethoxyphenyl)-2-methyloctahydro-1H-isoindol-5-amine, prepared in comparative example 6, (8.6 mg, 0.03 mmol) in THF (1.0 ml). Was treated with 4-iodobenzoyl chloride (0.04 mmol) and triethylamine (14 ul, 0.1 mmol). The mixture was stirred overnight then 2 drops of 1N NaOH were added. The solvent was evaporated and the residue purified by preparative HPLC using acetonitrile-water 20-50 containing 0.1% TFA.

The fractions were pooled and the mixture was evaporated, dissolved in chloroform, then washed with 1N NaOH. The organic phase was evaporated to give the title compound. Yield 6.0 mg (38%). White solid. HPLC purity 99%, MS (electrospray; [M+H]⁺) m/z: 521.2. HRMS for C₂₄H₂₉IN₂O₃: Calcd, 520.1223; found 520.1219.

### EXAMPLE 8

### N-[(3aS*,5R*,7aR*)-7a-(3,4-dimethoxyphenyl)-2-methyloctahydro-1H-isoindol-5-yl]-3,4-dimethoxybenzamide trifluoroacetate

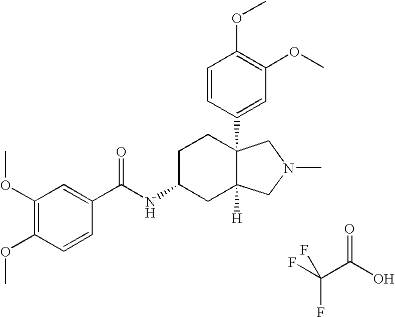

Procedure: as for example 7

Starting material: 3,4 dimethoxybenzoyl chloride.

Purified by reversed phase HPLC using acetonitrile-water 20-50 containing 0.1% TFA then evaporated to give the title compound. Yield 5.8 mg (34%). White solid. HPLC purity 99%, MS (electrospray; [M+H]⁺) m/z: 455.6. HRMS for C₂₆H₃₄N₂O₅: Calcd, 454.2468; found 454.2483.

### EXAMPLE 9

### 4-chloro-N-[(3aS*,5R*,7aR*)-7a-(3,4-dimethoxyphenyl)-2-methyloctahydro-1H-isoindol-5-yl]benzamide

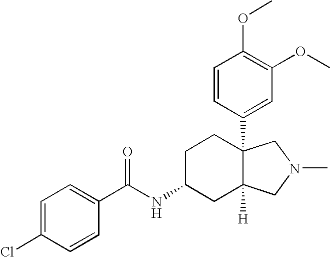

Procedure: as for example 7

Starting material: 4-chlorobenzoyl chloride.

Yield 8.3 mg (64%). White solid. HPLC purity 99%, MS (electrospray; [M+H]⁺) m/z: 429.0. HRMS for C₂₄H₂₉ClN₂O₃: Calcd, 428.1867; found 428.1882.

### EXAMPLE 10

### N-[(3aS*,5R*,7aR*)-7a-(3,4-dimethoxyphenyl)-2-methyloctahydro-1H-isoindol-5-yl]-4-fluorobenzamide

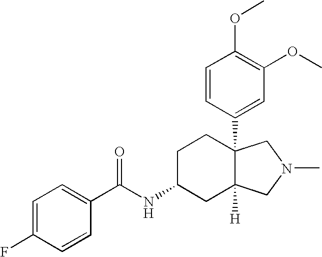

Procedure: as for example 7

Starting material: 4-fluorobenzoyl chloride.

Yield 2.7 mg (22%). White solid. HPLC purity 99%, MS (electrospray; [M+H]⁺) m/z: 413.5. HRMS for C₂₄H₂₉FN₂O₃: Calculated 412.2162, found 412.2175.

### EXAMPLE 11

### 4-bromo-N-[(3aS*,5R*,7aR*)-7a-(3,4-dimethoxyphenyl)-2-methyloctahydro-1H-isoindol-5-yl]benzamide

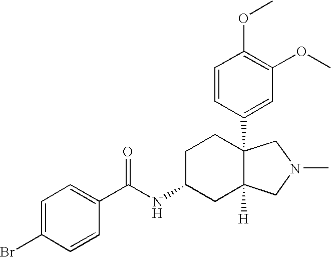

Procedure: as for example 7

Starting material: 4-bromobenzoyl chloride.

Yield 3.8 mg (27%). White solid. HPLC purity 99%, MS (electrospray; [M+H]⁺) m/z: 474.4. HRMS for C₂₄H₂₉BrN₂O₃: Calcd, 472.1362; found 472.1366.

### EXAMPLE 12

### N-[(3aS*,5R*,7aR*)-7a-(3,4-dimethoxyphenyl)-2-methyloctahydro-1H-isoindol-5-yl]-3-fluoro-4-(trifluoromethyl)benzamide

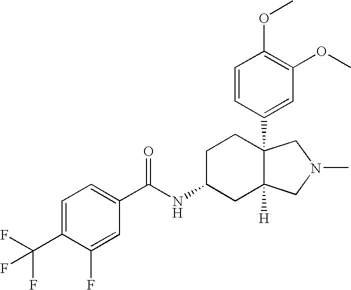

Procedure: as for example 7

Starting material: 3-fluoro-4-(trifluoromethyl)benzoyl chloride.

Yield 7.3 mg (51%). White solid. HPLC purity 99%, MS (electrospray; [M+H]⁺) m/z: 481.5. HRMS for C₂₅H₂₈F₄N₂O₃: Calcd, 480.2036; found 480.2051.

### EXAMPLE 13

### 4-bromo-N-[(3aS*,5R*,7aR*)-7a-(3,4-dimethoxyphenyl)-2-methyloctahydro-1H-isoindol-5-yl]-3-methylbenzamide

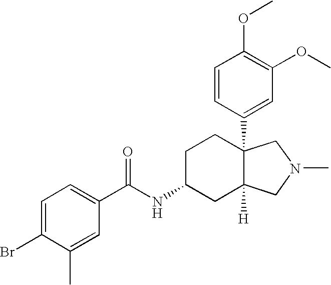

Procedure: as for example 7

Starting material: 4-bromo-3-methyl benzoyl chloride.

Yield 5.4 mg (37%). White solid. HPLC purity 99%, MS (electrospray; [M+H]⁺) m/z: 489.4. HRMS for C₂₅H₃₁BrN₂O₃: Calcd, 486.1518; found 486.1506.

### EXAMPLE 14

### N-[(3aS*,5R*,7aR*)-7a-(3,4-dimethoxyphenyl)-2-methyloctahydro-1H-isoindol-5-yl]-4-fluoro-3-(trifluoromethyl)benzamide

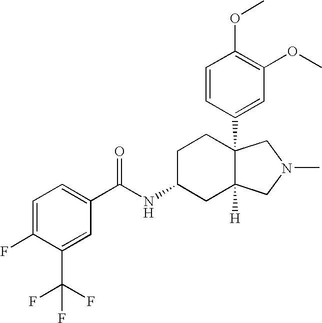

Procedure: as for example 7

Starting material: 4-fluoro-3-(trifluoromethyl)benzoyl chloride.

Yield 6.8 mg (47%). White solid. HPLC purity 99%, MS (electrospray; [M+H]⁺) m/z: 481.5. HRMS for C₂₅H₂₈F₄N₂O₃: Calcd, 480.2036; found 480.2027.

### EXAMPLE 15

### N-[(3aS*,5R*,7aR*)-7a-(3,4-dimethoxyphenyl)-2-methyloctahydro-1H-isoindol-5-yl]-4-(trifluoromethyl)benzamide

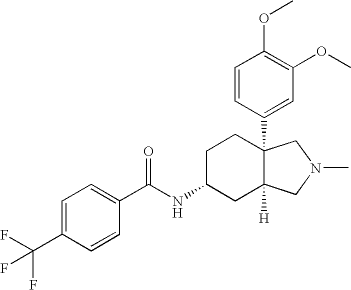

Procedure: as for example 7

Starting material: 4-(trifluoromethyl)benzoyl chloride.

Yield 4.0 mg (29%). White solid. HPLC purity 99%, MS (electrospray; [M+H]⁺) m/z: 481.5. HRMS for C₂₅H₂₉F₃N₂O₃: Calcd, 462.2130; found 462.2148.

### EXAMPLE 16

### N-[(3aS*,5R*,7aR*)-7a-(3,4-dimethoxyphenyl)-2-methyloctahydro-1H-isoindol-5-yl]-2,2-difluoro-1,3-benzodioxole-5-carboxamide

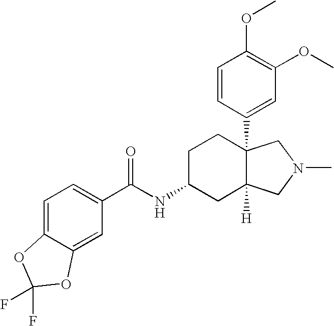

Procedure: as for example 7

Starting material: 2,2-Difluoro-1,3-benzodioxole-5-carbonyl chloride.

Yield 4.0 mg (29%). White solid. HPLC purity 99%, MS (electrospray; [M+H]⁺) m/z: 475.5. HRMS for C₂₅H₂₈F₂N₂O₅: Calcd, 474.1966, found 474.1985.

### EXAMPLE 17

### N-(3-chloro-4-fluorophenyl)-N′-[(3aS*,5R*,7aR*)-7a-(3,4-dimethoxyphenyl)-2-methyloctahydro-1H-isoindol-5-yl]urea trifluoroacetate

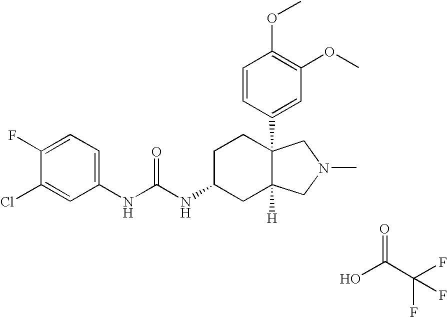

A solution of (3aS*,5R*,7aR*)-7a-(3,4-dimethoxyphenyl)-2-methyloctahydro-1H-isoindol-5-amine, prepared in comparative example 6, (8.6 mg, 0.03 mmol) in THF (1.0 ml) was treated with 3-chloro-4-fluorophenyl isocyanate (0.04 mmol). The mixture was stirred overnight then 2 drops of 1N NaOH were added. The solvent was evaporated and the residue purified by preparative HPLC using acetonitrile-water 20-50 containing 0.1% TFA.

The fractions were pooled and the mixture was evaporated to give the title compound. Yield 3.9 mg (23%). Colorless liquid. HPLC purity 99%, MS (electrospray; [M+H]⁺) m/z: 462.2. HRMS for C₂₄H₂₉ClFN₃O₃: Calcd, 461.1882, found 461.1885.

### EXAMPLE 18

### N-[4-chloro-3-(trifluoromethyl)phenyl]-N′-[(3aS*,5R*,7aR*)-7a-(3,4-dimethoxyphenyl)-2-methyloctahydro-1H-isoindol-5-yl]urea trifluoroacetate

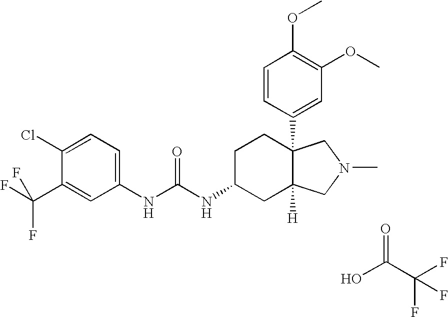

Procedure: as for example 17

Starting material: 4-chloro-3-(trifluoromethyl)phenyl isocyanate.

Yield 11.8 mg (63%). Light brown liquid. HPLC purity 95%, MS (electrospray; [M+H]⁺) m/z: 512.2. HRMS for C₂₅H₂₉ClF₃N₃O₃: Calcd, 511.1850, found 511.1869.

### EXAMPLE 19

### N-(3,5-difluorophenyl)-N′-[(3aS*,5R*,7aR*)-7a-(3,4-dimethoxyphenyl)-2-methyloctahydro-1H-isoindol-5-yl]urea trifluoroacetate

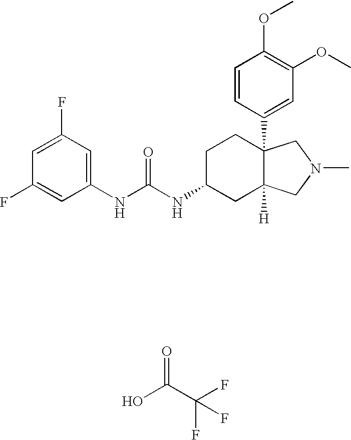

Procedure: as for example 17

Starting material: 3,5-difluorophenyl isocyanate.

Yield 8.3 mg (49%). Colorless liquid. HPLC purity 99%, MS (electrospray; [M+H]⁺) m/z: 446.2. HRMS for C₂₄H₂₉F₂N₃O₃: Calcd, 445.2177, found 445.2189.

### EXAMPLE 20

### N-[(3aS*,5R*,7aR*)-7a-(3,4-dimethoxyphenyl)-2-methyloctahydro-1H-isoindol-5-yl]-N′-(2,3,4-trifluorophenyl)urea trifluoroacetate

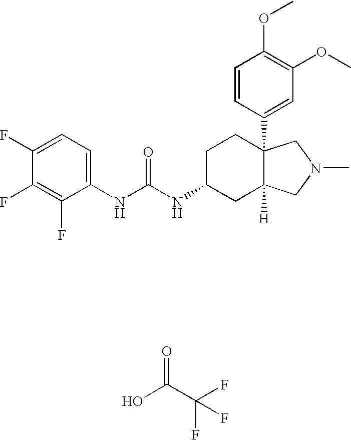

Procedure: as for example 17

Starting material: 2,3,4-trifluorophenyl isocyanate.

Yield 9.5 mg (55%). Colorless liquid. HPLC purity 90%, MS (electrospray; [M+H]⁺) m/z: 464.2. HRMS for C₂₄H₂₉F₃N₃O₃: Calcd, 463.2083, found 463.2077.

### EXAMPLE 21

### N-[(3aS*,5R*,7aR*)-7a-(3,4-dimethoxyphenyl)-2-methyloctahydro-1H-isoindol-5-yl]-N′-(3-fluorophenyl)urea trifluoroacetate

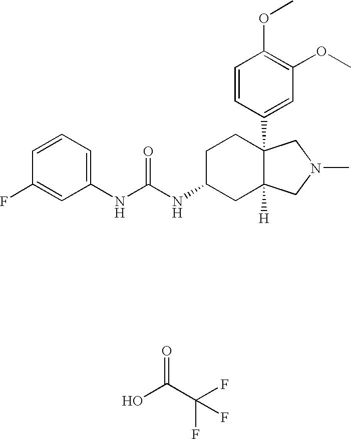

Procedure: as for example 17

Starting material: 3-fluorophenyl isocyanate.

Purified by reversed phase HPLC using acetonitrile-water 20-50 containing 0.1% TFA. The fractions were pooled and the mixture was evaporated. Isolated as the TFA-salt. Yield 5.6 mg (34%). Colorless liquid. HPLC purity 99%, MS (electrospray; [M+H]⁺) m/z: 428.2. HRMS for C₂₄H₃₀FN₃O₃: Calcd, 427.2271, found 427.2267.

### EXAMPLE 22

### N-(3-chlorophenyl)-N′-[(3 aS*,5R*,7aR*)-7a-(3,4-dimethoxyphenyl)-2-methyloctahydro-1H-isoindol-5-yl]urea trifluoroacetate

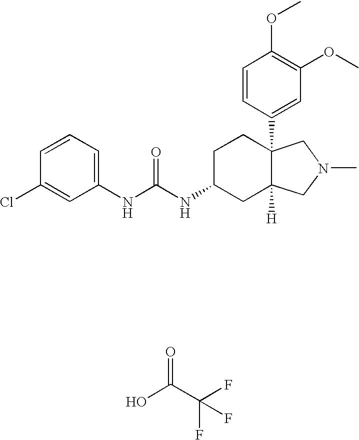

Procedure: as for example 17

Starting material: 3-chlorophenyl isocyanate.

Yield 6.4 mg (38%). Colorless liquid. HPLC purity 99%, MS (electrospray; [M+H]⁺) m/z: 444.2. HRMS for C₂₄H₃₀ClN₃O₃: Calcd, 443.1976, found 443.1979.

### EXAMPLE 23

### N-[(3aS*,5R*,7aR*)-7a-(3,4-dimethoxyphenyl)-2-methyloctahydro-1H-isoindol-5-yl]isonicotinamide 1-oxide trifluoroacetate

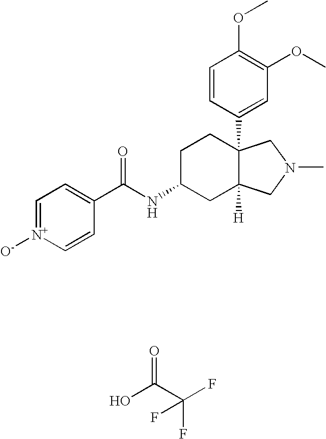

4-carboxypyridine-N-oxide (25 mg, 0.18 mmol), hydroxybenzotriazole (25 mg, 0.185 mmol), 1-[3-(dimethylaminopropyl)]-3-ethylcarbodiimide hydrochloride (35 mg, 0.185 mmol and triethylamine (35 mg, 0.35 mmol) were mixed in DMF (2 ml) followed by addition of (3aS*,5R*,7aR*)-7a-(3,4-dimethoxyphenyl)-2-methyloctahydro-1H-isoindol-5-amine, prepared in comparative example 6, (35 mg, 0.12 mmol).

The mixture was stirred for 2 hours then diluted with ether and washed twice with water. The aqueous layer was evaporated and the crude product was purified by reversed phase HPLC using acetonitrile-water 05-20 containing 0.1% TFA. To give the title compound. Yield 16.6 mg (26%). Colorless oil. HPLC purity 99%, MS (electronspray; [M+H]⁺) m/z: 412.2. HRMS for C₂₃H₂₉N₃O₄: Calcd, 411.2164, found 411.2158.

### EXAMPLE 24

### N-(3,4-difluorophenyl)-N′-[(3aS*,5R*,7aR*)-7a-(3,4-dimethoxyphenyl)-2-methyloctahydro-1H-isoindol-5-yl]urea

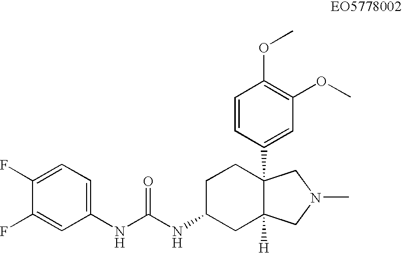

3,4-difluorophenylisocyanate (22 mg, 0.14 mmol) was added to a solution of (3aS*,5R*,7aR*)-7a-(3,4-dimethoxyphenyl)-2-methyloctahydro-1H-isoindol-5-amine, prepared in comparative example 6, (35 mg, 0.12 mmol) in DCM (2 mL). The mixture was stirred over night at room temperature. The solvent was evaporated and the crude product purified by flash chromatography (CHCl₃ sat. with NH₃ (g)/iso-hexane, 60:40, 70:30, 100:0). To give the title compound (6 mg, 11%). HPLC purity 96%, 1H NMR (500 MHz, CHLOROFORM-D) δ ppm 1.01 (qd, J=12.57, 12.24, 3.60 Hz, 1H) 1.38-1.46 (m, 1H) 1.76-1.84 (m, 1H) 1.98-2.05 (m, 1H) 2.03-2.10 (m, 1H) 2.12 (td, J=13.67, 3.30 Hz, 1H) 2.40 (s, 3H) 2.69 (d, J=9.28 Hz, 1H) 2.84 (t, J=8.91 Hz, 1H) 2.84 (d, J=9.40 Hz, 1H) 2.87-2.94 (m, 1H) 3.06 (t, J=8.91 Hz, 1H) 3.85 (s, 3H) 3.86 (s, 3H) 3.87-3.97 (m, 1H) 4.54 (d, J=7.93 Hz, 1H) 6.54 (s, 1H) 6.81 (d, J=8.42 Hz, 1H) 6.84 (d, J=2.20 Hz, 1H) 6.86-6.90 (m, 1H) 6.88 (dd, J=8.30, 2.20 Hz, 1H) 7.02 (dt, J=9.89, 8.79 Hz, 1H) 7.32 (ddd, J=12.21, 7.08, 2.69 Hz, 1H). MS (ESI+) for 446 m/z C₂₄H₂₉F₂N₃O₃ (M+H)⁺.

### EXAMPLE 25

### N-[(3 aS*,5R*,7aR*)-7a-(3,4-dimethoxyphenyl)-2-methyloctahydro-1H-isoindol-5-yl]-N′-[3-fluoro-5-(trifluoromethyl)phenyl]urea acetate

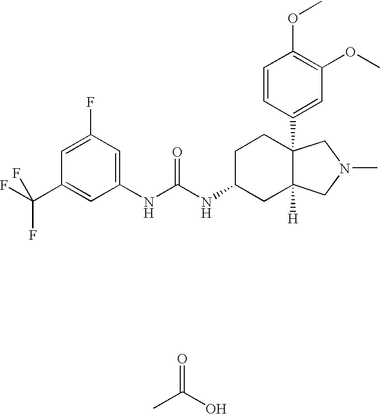

3-fluoro-5-(trifluoromethyl)phenylisocyanate (17 mg, 0.083 mmol) was added to a solution of (3 aS*,5R*,7aR*)-7a-(3,4-dimethoxyphenyl)-2-methyloctahydro-1H-isoindol-5-amine, prepared in comparative example 6, (20 mg, 0.069 mmol) in DCM (1 mL). The reaction was stirred over night for 16 hours. Solvent was evaporated and the crude product was purified by preparative HPLC (ACE-column 30*150 mm, MeCN 30-65%, ammonium acetate buffer) to give the product (3 mg, 9%) as an off-white solid. HPLC purity 91%, 1H NMR (400 MHz, CHLOROFORM-D) δ ppm 1.04-1.16 (m, 1H) 1.55 (s, 1H) 1.77-1.85 (m, 1H) 1.95-2.07 (m, 2H) 2.09-2.17 (m, 1H) 2.68 (s, 3H) 2.98-3.05 (m, J=11.54 Hz, 1H) 3.07-3.17 (m, 1H) 3.29 (s, 2H) 3.55-3.64 (m, 1H) 3.84 (s, 3H) 3.84 (s, 3H) 3.87-3.88 (m, 1H) 5.65 (br. s., 1H) 6.76-6.78 (m, 1H) 6.78-6.81 (m, 2H) 6.84-6.88 (m, 1H) 7.40-7.43 (m, 1H) 7.46-7.54 (m, 1H) 8.38 (s, 1H). MS (ESI+) for C₂₅H₂₉F₄N₃O₃ m/z 496 (M+H)⁺. HRMS Calc: 495.2145; Found: 495.2144

### EXAMPLE 26

### N-[(3aS*,5R*,7aR*)-7a-(3,4-dimethoxyphenyl)-2-methyloctahdro-1H-isoindol-5-yl]-N′-[2-fluoro-3-(trifluoromethyl)phenyl]urea acetate

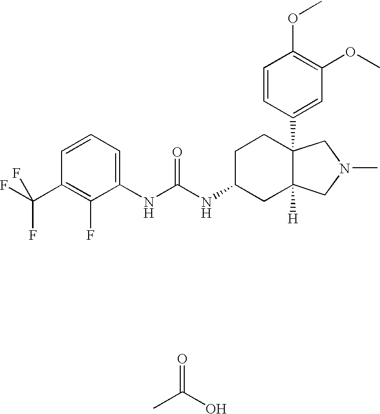

Procedure: as for example 25

Starting material: 2-fluoro-3-(trifluoromethyl)phenylisocyanate

Yield: 9 mg, 18%.

HPLC purity 90%, 1H NMR (400 MHz, CHLOROFORM-D) δ ppm 1.13 (s, 1H) 1.45-1.55 (m, 1H) 1.80-1.88 (m, 1H) 1.97-2.08 (m, 2H) 2.13-2.21 (m, 1H) 2.64 (s, 3H) 3.08 (s, 2H) 3.15-3.21 (m, 1H) 3.22-3.30 (m, 1H) 3.35-3.43 (m, 1H) 3.84 (s, 6H) 3.89 (s, 1H) 5.60-5.65 (m, 1H) 6.79 (d, J=8.28 Hz, 1H) 6.81-6.82 (m, 1H) 6.83-6.87 (m, 1H) 7.10-7.16 (m, 2H) 7.45 (s, 1H) 8.30-8.40 (m, 1H). MS (ESI+) for C₂₅H₂₉F₄N₃O₃ m/z 496 (M+H)⁺. HRMS Calc: 495.2145; Found: 495.140

### EXAMPLE 27

### N-(2,5-difluorophenyl)-N′-[(3aS*,5R*,7aR*)-7a-(3,4-dimethoxyphenyl)-2-methyloctahydro-1H-isoindol-5-yl]urea

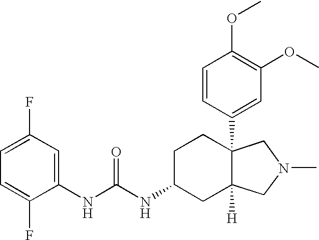

Procedure: as for example 25

Starting material: 2,5-difluorophenylisocyanate

The product was purified by preparative HPLC then this material was extracted twice with 1M aq. NaOH and DCM to get rid of acetic acid. The organic layers were combined, dried and concentrated. This yielded 17 mg, 27%. HPLC purity 99%, 1H NMR (400 MHz, CHLOROFORM-D) δ ppm 0.99-1.11 (m, 1H) 1.43-1.52 (m, 1H) 1.79-1.86 (m, 1H) 2.00-2.19 (m, 3H) 2.40 (s, 3H) 2.69 (d, J=9.29 Hz, 1H) 2.84 (t, J=8.91 Hz, 3H) 3.07 (t, J=8.66 Hz, 1H) 3.85 (s, 3H) 3.85 (s, 3H) 3.89-3.99 (m, 1H) 4.64 (d, J=7.53 Hz, 1H) 6.53-6.62 (m, 2H) 6.80 (d, J=8.53 Hz, 1H) 6.82 (d, J=2.01 Hz, 1H) 6.85-6.89 (m, 1H) 6.89-6.99 (m, 1H) 7.90-8.00 (m, 1H). MS (ESI+) for C₂₄H₂₉F₂N₃O₃ m/z 446 (M+H)⁺.

### EXAMPLE 28

### 3,4-dichloro-N-[(3aS*,5R*,7aR*)-7a-(3,4-dimethoxyphenyl)-2-methyloctahydro-1H-isoindol-5-yl]benzamide

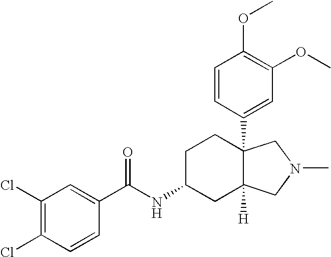

NEt₃ (31 μL, 0.22 mmol) and 3,4-dichlorobenzoyl chloride (28 mg, 0.13 mmol) were added to a solution of (3aS*,5R*,7aR*)-7a-(3,4-dimethoxyphenyl)-2-methyloctahydro-1H-isoindol-5-amine, prepared in comparative example 6, (32 mg, 0.11 mmol) in DCM (1.5 mL). The mixture was stirred at rt. for 1.5 hours. Purification as in example 27, afforded 9 mg, 18%. HPLC purity 90%, 1H NMR (270 MHz, CHLOROFORM-D) δ ppm 1.08-1.21 (m, 1H) 1.52-1.66(m, 1H) 1.83-1.94(m, 1H) 2.01-2.11 (m, 1H) 2.11-2.21 (m, 2H) 2.42 (s, 3H) 2.70 (d, J=9.15 Hz, 1H) 2.83-3.02 (m, 3H) 3.12 (t, J=8.04 Hz, 1H) 3.86 (s, 3H) 3.88 (s, 3H) 4.16-4.33 (m, 1H) 5.76 (d, J=7.67 Hz, 1H) 6.82 (d, J=8.41 Hz, 1H) 6.85 (d, J=1.98 Hz, 1H) 6.88-6.94 (m, 1H) 7.42-7.54 (m, 2H) 7.76 (d, J=1.73 Hz, 1H). MS (ESI+) for C₂₄H₂₈Cl₂N₂O₃ m/z 463 (Monoiso. Mass+H)⁺. HRMS Calc: 462.1477; Found: 462.1484

### EXAMPLE 29

### N-[(3aS*,5R*,7aR*)-7a-(3,4-dimethoxyphenyl)-2-methyloctahydro-1H-isoindol-5-yl]-N′-(3,4,5-trifluorophenyl)urea

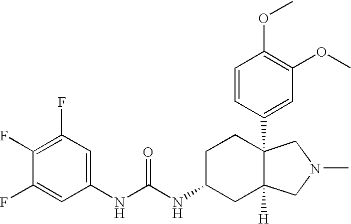

Triphosgene (17 mg, 0.058 mmol) in DCM (1 mL) was, added dropwise under N₂, to a solution of (3aS*,5R*,7aR*)-7a-(3,4-dimethoxyphenyl)-2-methyloctahydro-1H-isoindol-5-amine, prepared in comparative example 6, (42 mg, 0.14 mmol) and dry NEt₃ (40 μL, 0.28 mmol) in DCM (2 mL). The solution was stirred at rt. for 3 hours, then 3,4,5-trifluoroaniline (21 mg, 0.14 mmol) was added and stirring continued over night. Purification and work-up as for example 27, afforded the product as the free base (8 mg, 12%). HPLC purity 100%, 1H NMR (400 MHz, CHLOROFORM-D) δ ppm 0.91-1.04 (m, 1H) 1.34-1.44 (m, 1H) 1.77-1.81 (m, 1H) 1.96-2.15 (m, 3H) 2.39 (s, 3H) 2.67 (d, J=9.29 Hz, 1H) 2.82 (dd, J=9.16, 7.40 Hz, 3H) 3.03 (t, J=8.53 Hz, 1H) 3.83 (s, 3H) 3.84 (s, 3H) 3.87-3.95 (m, 1H) 4.66 (d, J=7.78 Hz, 1H) 6.78-6.84 (m, 2H) 6.85-6.89 (m, 1H) 6.99-7.06 (m, 2H). MS (ESI+) for C₂₄H₂₈F₃N₃O₃ m/z 464 (M+H)⁺. HRMS Calc: 463.2083; Found: 463.2088

### COMPARATIVE EXAMPLE 30

### (3aS*,5S*,7aR*)-7a-(3,4-dimethoxyphenyl)-2-methyloctahydro-1H-isoindol-5-amine

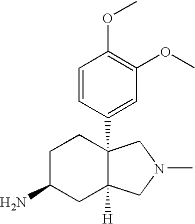

(3aS*,7aR*)-7a-(3,4-dimethoxyphenyl)-2-methyloctahydro-5H-isoindol-5-one, prepared in comparative example 5, (132 mg, 0.45 mmol) was dissolved in dry methanol (25 mL). Ammonium acetate (3 eq) was added and the mixture was stirred at 65° C. for 30 minutes, whereafter NaCNBH₃ (1.2 eq) was added. The reaction was stirred an additional 15 min with continous stirring. The solvent was evaporated and the residue was partitioned between 1M aq. NaOH (20 mL) and DCM (20 mL). The aq. layer was extracted once more with DCM and the organic layers were combined, dried and evaporated to give the crude product which was purified by flash-chromatography over silica (CHCl₃ sat. with NH₃ (g)/iso-hexane, 60:40) to give the title compound (11 mg, 8%). HPLC purity 90%, 1H NMR (500 MHz, CHLOROFORM-D) δ ppm 1.30-1.39 (m, 1H) 1.40 (dt, J=13.18, 10.86, 10.62 Hz, 1H) 1.66-1.73 (m, 1H) 1.70-1.77 (m, 1H) 1.85-1.93 (m, J=13.08, 5.72, 4.09, 1.28 Hz, 1H) 1.98-2.06 (m, 1H) 2.41 (s, 3H) 2.50 (dd, J=9.46, 2.38 Hz, 1H) 2.51-2.58 (m, 1H) 2.80-2.88 (m, 1H) 2.91 (dd, J=9.40, 6.59 Hz, 1H) 2.91-2.95 (m, J=9.64 Hz, 1H) 3.04 (d, J=9.64 Hz, 1H) 3.86 (s, 3H) 3.89 (s, 3H) 6.81 (d, J=8.42 Hz, 1H) 6.91 (d, J=2.20 Hz, 1H) 6.94 (dd, J=8.30, 2.32 Hz, 1H). MS (ESI+) for C₁₇H₂₆N₂O₂ m/z 291 (M+H)⁺. The relative stereochemistry of this structure was further confirmed by NOE experiment.

### EXAMPLE 31

### N-[(3aS*,5S*,7aR*)-7a-(3,4-dimethoxyphenyl)-2-methyloctahydro-1H-isoindol-5-yl]-N′-[2-fluoro-3-(trifluoromethyl)phenyl]urea acetate

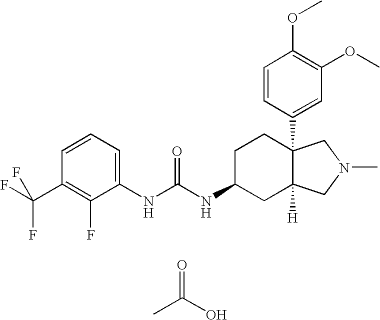

2-fluoro-3-(trifluoromethyl)phenylisocyanate (16 mg, 0.079 mmol) was added to a solution of (3aS*,5S*,7aR*)-7a-(3,4-dimethoxyphenyl)-2-methyloctahydro-1H-isoindol-5-amine, prepared in comparative example 30, (20 mg, 0.069 mmol) in DCM (1 mL). The reaction was stirred over night for 16 hours. Solvent was evaporated and the crude product was purified by preparative HPLC (ACE-column 30×150 mm, MeCN 30-65%, ammonium acetate buffer) to give 19 mg, 60%. HPLC purity 95%, 1H NMR (400 MHz, CHLOROFORM-D) δ ppm 1.39-1.49 (m, 1H) 1.53-1.64 (m, J=12.55 Hz, 2H) 1.90-2.07 (m, 2H) 2.25-2.34 (m, 1H) 2.68 (s, 3H) 2.75 (d, J=11.04 Hz, 1H) 3.00 (t, J=11.17 Hz, 1H) 3.05-3.13 (m, 1H) 3.71 (d, J=11.04 Hz, 1H) 3.87 (s, 6H) 4.13-4.19 (m, 1H) 4.41-4.48 (m, 1H) 6.75-6.76 (m, 1H) 6.83-6.86 (m, 2H) 7.06-7.17 (m, 2H) 7.81-7.87 (m, 1H) 8.51-8.58 (m, 1H) 8.60-8.68 (m, 1H). HRMS Calc: 495.2145; Found: 495.2143

### EXAMPLE 32

### 3,4-dichloro-N-[(3aS*,5S*,7aR*)-7a-(3,4-dimethoxyphenyl)-2-methyloctahydro-1H-isoindol-5-yl]benzamide

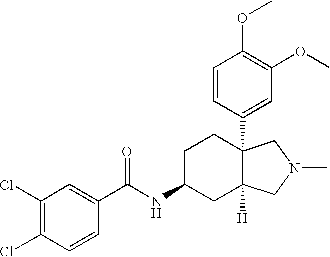

Procedure: as for example 28.

Starting material: (3aS*,5S*,7aR*)-7a-(3,4-dimethoxyphenyl)-2-methyloctahydro-1H-isoindol-5-amine prepared in comparative example 30, (25 mg; 0.086 mmol)

Yield 8 mg. HPLC purity 96%, 1H NMR (270 MHz, METHANOL-D4) δ ppm 1.42-1.64 (m, 3H) 1.65-1.74 (m, 1H) 1.94-2.05 (m, 2H) 2.57 (s, 3H) 2.95-3.11 (m, 2H) 3.16-3.22 (m, 1H) 3.61-3.67 (m, 2H) 3.67 (s, 3H) 3.72 (s, 3H) 3.78-3.91 (m, 1H) 6.79-6.84 (m, 1H) 6.90-6.96 (m, 2H) 7.48 (d, J=8.41 Hz, 1H) 7.58-7.65 (m, 1H) 7.85 (d, J=1.98 Hz, 1H). MS (ESI+) for C₂₄H₂₈Cl₂N₂O₃ m/z 463 (Monoiso. Mass+H)⁺. HRMS Calc: 462.1477; Found: 462.1471

### EXAMPLE 33

### N-[(3aS*,5R*,7aR*)-7a-(3,4-dimethoxyphenyl)-2-methyloctahydro-1H-isoindol-5-yl]-N′-[4-fluoro-3-(trifluoromethyl)phenyl]urea trifluoroacetate

A solution of (3aS*,5R*,7aR*)-7a-(3,4-dimethoxyphenyl)-2-methyloctahydro-1H-isoindol-5-amine, prepared in comparative example 6, (11.2 mg; 0.038 mmol) in THF (2.0 ml) was treated with the 4-fluoro-3-(trifluoromethyl)phenylisocyanate (1 eq) and the mixture was allowed to stand at RT overnight. The solvent was evaporated and the residues purified by HPLC (YMC column—gradient 20-60 MeCN) to afford the title compound (10.9 mg) as a colourless solid. MS (ESI+) for C₂₄H₂₈Cl₂N₂O₃ m/z 466 (Monoiso. Mass+H)⁺.

### EXAMPLE 34 AND EXAMPLE 35

### N-[(3aS,5R,7aR)-7a-(3,4-dimethoxyphenyl)-2-methyloctahydro-1H-isoindol-5-yl]-N′-[4-fluoro-3-(trifluoromethyl)phenyl]urea and N-[(3aR,5S,7aS)-7a-(3,4-dimethoxyphenyl)-2-methyloctahydro-1H-isoindol-5-yl]-N′-[4-fluoro-3-(trifluoromethyl)phenyl]urea

N-[(3aS*,5R*,7aR*)-7a-(3,4-dimethoxyphenyl)-2-methyloctahydro-1H-isoindol-5-yl]-N′-[4-fluoro-3-(trifluoromethyl)phenyl]urea trifluoroacetate, prepared in example 33, (10.0 mg) was resolved into separate enantiomers by chiral high-performance column chromatography.

Column: Chirobiotic V (250×6 mm), 5 microns

Mobile Phase: MeOH/AcOH/Triethylamine (100/0.1/0.1)

Flow Rate: 5 ml/min

Run Time: 30 min

Temp: 21° C.

The first eluted peak was collected and the solvent evaporated to give 1.1 mg of a colourless solid which was arbitrarily assigned as N-[(3aS,5R,7aR)-7a-(3,4-dimethoxyphenyl)-2-methyloctahydro-1H-isoindol-5-yl]-N′-[4-fluoro-3-(trifluoromethyl)phenyl]urea, HRMS Calc for C₂₅H₂₉F₄N₃O₃: 495.2145, HRMS Found: 495.2125

The second eluted peak was collected and the solvent evaporated to give 3.3 mg of a colourless solid which was arbitrarily assigned as N-[(3aR,5S,7aS)-7a-(3,4-dimethoxyphenyl)-2-methyloctahydro-1H-isoindol-5-yl]-N′-[4-fluoro-3-(trifluoromethyl)phenyl]urea, HRMS Calc for C₂₅H₂₉F₄N₃O₃: 495.2145, HRMS Found: 495.2142

### EXAMPLE 36

### N-[(3aS*,5R*,7aR*)-7a-(3,4-Dimethoxyphenyl)-2-methyloctahydro- H-isoindol-5-yl]-3-fluorobenzamide trifluoroacetate

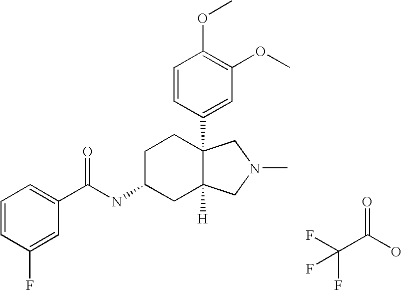

3-fluorobenzoyl chloride (7 mg, 0.0461 mmol) was added a solution of (3aS*,5R*,7aR*)-7a-(3,4-dimethoxyphenyl)-2-methyloctahydro-1H-isoindol-5-amine, prepared in comparative example 6, (13 mg, 0.0461 mmol) in DCM (1 ml) and triethylamine (8 μl, 0.0538 mmol). The mixture was stirred overnight then the solvent was evaporated. The residue was purified by preparative HPLC to yield 1.8 mg of the desired TFA-salt. Yield 7%, HPLC purity 98%, m/z 413 (M+H)⁺, HRMS for C₂₄H₂₉FN₂O₃: calcd: 412.2162, found: 412.2162.

### EXAMPLE 37

### 3-Chloro-N-[(3aS*,5R*,7aR*)-7a-(3,4-dimethoxyphenyl)-2-methyloctahydro-1H-isoindol-5-yl]benzamide trifluoroacetate

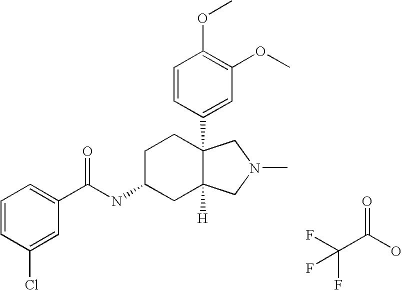

Procedure: as for example 36

Starting material: 3-chlorobenzoyl chloride

Yield 15%, HPLC purity 99%, m/z 429 (M+H)⁺, HRMS for C₂₄H₂₉ClN₂O₃: calcd: 428.1867, found: 428.1851.

### EXAMPLE 38

### 3-Bromo-N-[(3aS*,5R*,7aR*)-7a-(3,4-dimethoxyphenyl)-2-methyloctahydro-1H-isoindol-5-yl]benzamide trifluoroacetate

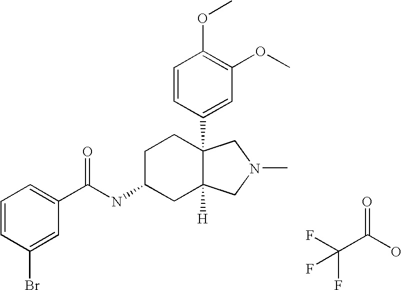

Procedure: as for example 36

Starting material: 3-bromobenzoyl chloride

Yield 17%, HPLC purity 99%, m/z 473 (M+H)⁺, HRMS for C₂₄H₂₉BrN₂O₃: calcd: 472.1362, found: 472.1362. 1H NMR (270 MHz, METHANOL-D₃) δ ppm 0.87-1.08 (m, 1H) 1.32-1.49 (m, 1H) 1.54-1.68 (m, 1H) 1.79-1.93 (m, 1H) 1.99-2.09 (m, 2H) 2.39 (s, 3H) 2.63 (d, J=9.15 Hz, 1H) 2.73-3.06 (m, 5H) 3.85 (s, 3H) 3.86 (s, 3H) 6.73-6.96 (m, 3H) 7.29-7.41 (m, 1H) 7.64-7.70 (m, 1H) 7.71-7.78 (m, 1H) 7.93-7.98 (m, 1H).

### EXAMPLE 39

### 3-[(Difluoromethyl)thiol-N-[(3aS*,5R*,7aR*)-7a-(3,4-dimethoxyphenyl)-2-methyloctahydro-1H-isoindol-5-yl]benzamide trifluoroacetate

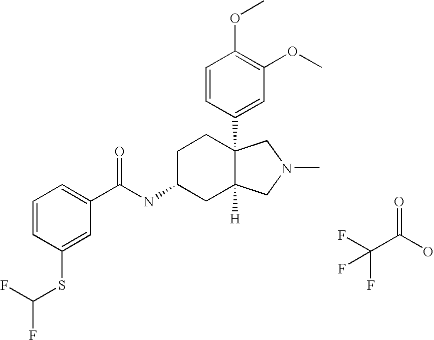

Procedure: as for example 36

Starting material: 3-difluoromethylthiobenzoyl chloride

Yield 14%, HPLC purity 100%, m/z 477 (M+H)⁺, HRMS for C₂₅H₃₀F₂N₂O₃S: calcd: 476.1945, found: 476.1942.

### EXAMPLE 40

### 3-Cyano-N-[(3aS*,5R*,7aR*)-7a-(3,4-dimethoxyphenyl)-2-methyloctahydro-1H-isoindol-5-yl]benzamide trifluoroacetate

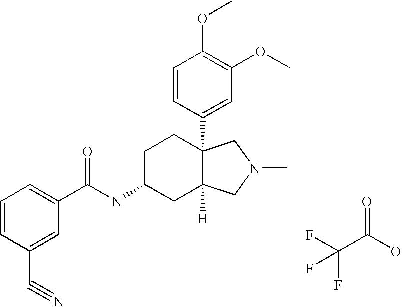

Procedure: as for example 36

Starting material: 3-cyanobenzoyl chloride

Yield 12%, HPLC purity 100%, m/z 420 (M+H)⁺, HRMS for C₂₅H₂₉N₃O₃: calcd: 419.2209, found: 419.2202.

### EXAMPLE 41

### 4-Cyano-N-[(3aS*,5R*,7aR*)-7a-(3,4-dimethoxyphenyl)-2-methyloctahydro-1H-isoindol-5-yl]benzamide trifluoroacetate

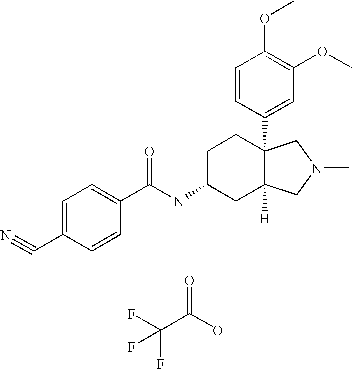

Procedure: as for example 36

Starting material: 4-cyanobenzoyl chloride

Yield 14%, HPLC purity 96%, m/z 420 (M+H)⁺, HRMS for C₂₅H₂₉N₃O₃: calcd: 419.2209, found: 419.2201, 1H NMR (270 MHz, METHANOL-D₃) δ ppm 0.87-1.08 (m, 1H) 1.32-1.49 (m, 1H) 1.54-1.68 (m, 1H) 1.79-1.93 (m, 1H) 1.99-2.09 (m, 2H) 2.39 (s, 3H) 2.63 (d, J=9.15 Hz, 1H) 2.73-3.06 (m, 5H) 3.85 (s, 3H) 3.86 (s, 3H) 6.73-6.96 (m, 3 H) 7.74-7.96 (m, 4H).

### EXAMPLE 42

### N-[(3 aS*,5R*,7aR*)-7a-(3,4-Dimethoxyphenyl)-2-methyloctahydro-1H-isoindol-5-yl]-3,4-difluorobenzamide trifluoroacetate

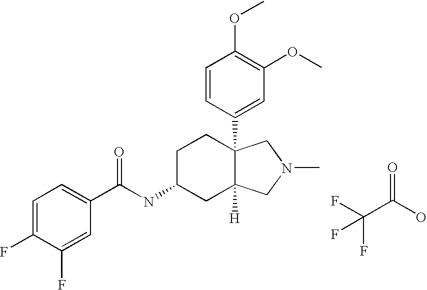

Procedure: as for example 36

Starting material: 3,4-difluorobenzoyl chloride

Yield 11%, HPLC purity 97%, m/z 431 (M+H)⁺, HRMS for C₂₄H₂₈F₂N₂O₃: calcd: 430.2068, found: 430.2087.

### EXAMPLE 43

### N-[(3aS*,5R*,7aR*)-7a-(3,4-Dimethoxyphenyl)-2-methyloctahydro-1H-isoindol-5-yl]-3-fluoro-5-(trifluoromethyl)benzamide trifluoroacetate

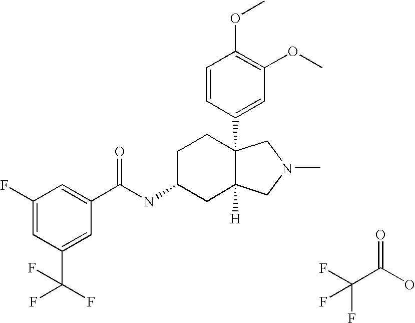

Procedure: as for example 36

Starting material: 3-fluoro-5-(trifluoromethyl)benzoyl chloride

Yield 10%, HPLC purity 100%, m/z 481 (M+H)⁺, HRMS for C₂₅H₂₈F₄N₂O₃: calcd: 480.2036, found: 480.2040.

### EXAMPLE 44

### N-[(3aS*,5R*,7aR*)-7a-(3,4-Dimethoxyphenyl)-2-methyloctahydro-1H-isoindol-5-yl]-2-fluoro-4-(trifluoromethyl)benzamide trifluoroacetate

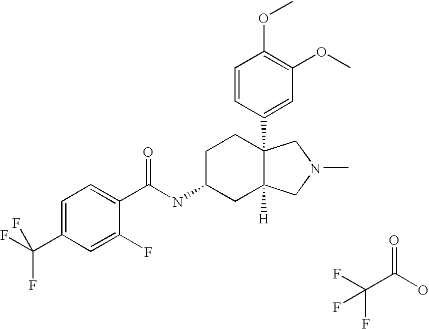

Procedure: as for example 36

Starting material: 2-fluoro-4-(trifluoromethyl)benzoyl chloride

Yield 18%, HPLC purity 98%, m/z 481 (M+H)⁺, HRMS for C₂₅H₂₈F₄N₂O₃: calcd: 480.2036, found: 480.2026, 1H NMR (270 MHz, METHANOL-D₃) δ ppm 0.87-1.08 (m, 1H) 1.32-1.49 (m, 1H) 1.54-1.68 (m, 1H) 1.79-1.93 (m, 1H) 1.99-2.09 (m, 2H) 2.39 (s, 3H) 2.63 (d, J=9.15 Hz, 1H) 2.73-3.06 (m, 5H) 3.85 (s, 3H) 3.86 (s, 3H) 6.73-6.96 (m, 3H) 7.49-7.81 (m, 3H).

### EXAMPLE 45

### N-(3aS*,5R*,7aR*)-7a-(3,4-Dimethoxyphenyl)-2-methyloctahydro-1H-isoindol-5-yl]-4-[(trifluoromethyl)thio]benzamide trifluoroacetate

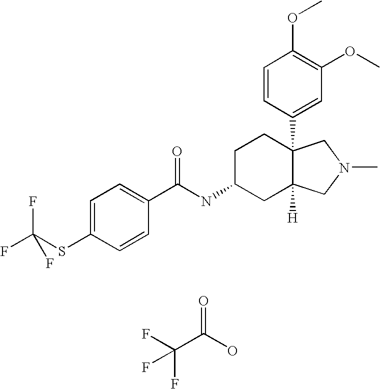

Procedure: as for example 36

Starting material: 4-(trifluoromethylthio)benzoyl chloride

Yield 15%, HPLC purity 97%, m/z 495 (M+H)⁺, HRMS for C₂₅H₂₉F₃N₂O₃S: calcd: 494.1851, found: 494.1857, 1H NMR (270 MHz, METHANOL-D₃) δ ppm 0.87-1.08 (m, 1H) 1.32-1.49 (m, 1H) 1.54-1.68 (m, 1H) 1.79-1.93 (m, 1H) 1.99-2.09 (m, 2H) 2.39 (s, 3H) 2.63 (d, J=9.15 Hz, 1H) 2.73-3.06 (m, 5H) 3.85 (s, 3H) 3.86 (s, 3H) 6.73-6.96 (m, 3H) 7.71-7.89 (m, 4H).

### EXAMPLE 46

### 3-Chloro-N-[(3aS*,5R*,7aR*)-7a-(3,4-dimethoxyphenyl)-2-methyloctahydro-1H-isoindol-5-yl]-2-fluorobenzamide trifluoroacetate

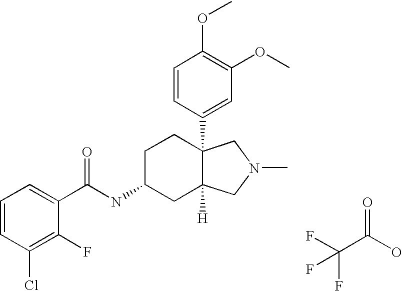

Procedure: as for example 36

Starting material: 3-chloro-2-fluorobenzoyl chloride

Yield 12%, HPLC purity 97%, m/z 447 (M+H)⁺, HRMS for C₂₄H₂₈ClFN₂O₃: calcd: 446.1772, found: 446.1768.

### EXAMPLE 47

### N-[(3aS*,5R*,7aR*)-7a-(3,4-Dimethoxyphenyl)-2-methyloctahydro-1H-isoindol-5-yl]-2,5-difluorobenzamide trifluoroacetate

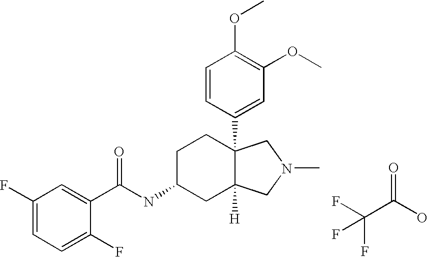

Procedure: as for example 36

Starting material: 2,5-difluorobenzoyl chloride

Yield 15%, HPLC purity 99%, m/z 431 (M+H)⁺, HRMS for C₂₄H₂₈F₂N₂O₃: calcd: 430.2068, found: 430.2052

### EXAMPLE 48

### N-[(3 aS*,5R*,7aR*)-7a-(3,4-Dimethoxyphenyl)-2-methyloctahydro-1H-isoindol-5-yl]-N′-(3,5-dimethylphenyl)urea trifluoroacetate

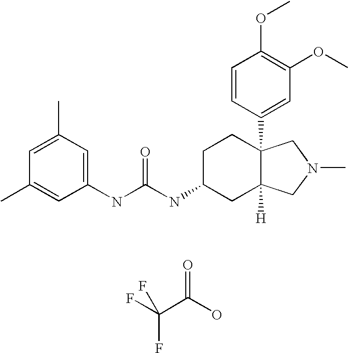

1-isocyanato-3,5-dimethylbenzene (7 mg, 0.0454 mmol) was added a solution of (3aS*,5R*,7aR*)-7a-(3,4-dimethoxyphenyl)-2-methyloctahydro-1H-isoindol-5-amine, prepared in comparative example 6, (12 mg, 0.0413 mmol) in DCM (1 ml). The mixture was stirred overnight and the solvent evaporated. The residue was purified by preparative HPLC to yield 3.0 mg of the desired TFA-salt. Yield 13%, HPLC purity 91%, m/z 438 (M+H)⁺, HRMS for C₂₆H₃₅N₃O₃: calcd: 437.2678, found: 437.2688.

### EXAMPLE 49

### N-(2,5-Dichlorophenyl)-N′-[(3aS*,5R*,7aR*)-7a-(3,4-dimethoxyphenyl)-2-methyloctahydro-1H-isoindol-5-yl]urea trifluoroacetate

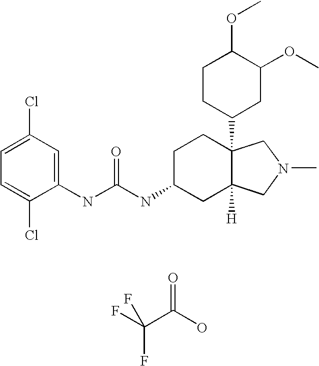

Procedure: as for example 48

Starting material: 2,5-dichlorophenylisocyanate

Yield 18%, HPLC purity 90%, m/z 478 (M+H)⁺, HRMS for C₂₄H₂₉Cl₂N₃O₃: calcd: 477.1586, found: 477.1586.

### EXAMPLE 50

### N-(2,4-Dichlorophenyl)-N′-[(3aS*,5R*,7aR*)-7a-(3,4-dimethoxyphenyl)-2-methyloctahydro-1H-isoindol-5-yl]urea trifluoroacetate

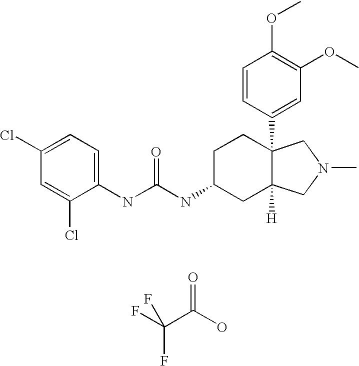

Procedure: as for example 48

Starting material: 2,4-dichlorophenylisocyanate

Yield 38%, HPLC purity 98%, m/z 478 (M+H)⁺, HRMS for C₂₄H₂₉Cl₂N₃O₃: calcd: 477.1586, found: 477.1588.

### EXAMPLE 51

### N-[(3aS*,5R*,7aR*)-7a-(3,4-Dimethoxyphenyl)-2-methyloctahydro-1H-isoindol-5-yl]-N′-(4-iodophenyl)urea trifluoroacetate

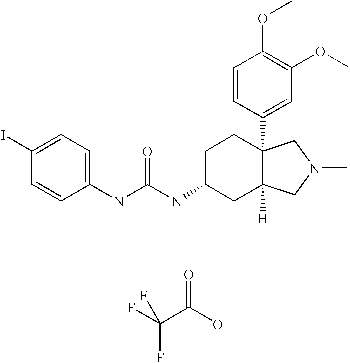

Procedure: as for example 48

Starting material: 4-iodophenylisocyanate

Yield 39%, HPLC purity 100%, m/z 536 (M+H)⁺, HRMS for C₂₄H₃₀IN₃O₃: calcd: 535.1332, found: 535.1313, 1H NMR (400 MHz, DMSO-D6, 70° C. due to rotamers) δ ppm 0.93-1.14 (m, 1H) 1.46-1.64 (m, 1H) 1.68-1.84 (m, 1H) 1.89-2.24 (m, 3H) 2.91 (s, 3H) 3.00-3.40 (m, 6H) 3.78 (s, 3H) 3.82 (s, 3H) 6.95-7.05 (m, 3H) 7.29 (dd, J=9.03, 2.44 Hz, 1H) 7.48 (d, J=2.44 Hz, 1H) 8.10-8.18 (m, J=8.91 Hz, 1H).

### EXAMPLE 52 AND EXAMPLE 53

### N-[(3aS,5R,7aR)-7a-(3,4-dimethoxyphenyl)-2-methyloctahydro-1H-isoindol-5-yl]-3-fluoro-4-(trifluoromethyl)benzamide trifluoroacetate and N-[(3aR,5S,7aS)-7a-(3,4-dimethoxyphenyl)-2-methyloctahydro-1H-isoindol-5-yl]-3-fluoro-4-(trifluoromethyl)benzamide trifluoroacetate

A sample of N-[(3aS*,5R*,7aR*)-7a-(3,4-dimethoxyphenyl)-2-methyloctahydro-1H-isoindol-5-yl]-3-fluoro-4-(trifluoromethyl)benzamide (17 mg), was prepared as in example 12, and was resolved by chiral HPLC in an analogous manner to the resolution carried out in examples 34 and 35.

The first eluted peak was collected and the solvent evaporated to give 1.2 mg of a colourless solid. The absolute stereochemistry was arbitrarily assigned as N-[(3aS,5R,7aR)-7a-(3,4-dimethoxyphenyl)-2-methyloctahydro-1H-isoindol-5-yl]-N′-[4-fluoro-3-(trifluoromethyl)phenyl]urea, 1H NMR (500 MHz, DMSO-D6) δ ppm 1.2 (s, 1 H) 1.7 (m, 2H) 1.9 (m, 1H) 2.0 (s, 1H) 2.2 (t, J=14.1 Hz, 1H) 2.9 (m, 2H) 2.9 (m, 2H) 3.3 (s, 3H) 3.6 (d, J=8.8 Hz, 1H) 3.7 (m, 1H) 3.8 (s, 3H) 3.8 (d, J=1.6 Hz, 3H) 7.0 (s, 3 H) 7.9 (s, 3H) 8.5 (s, 1H). HRMS Calc for C₂₅H₂₈F₄N₂O₃: 480.2036, HRMS Found: 480.2023, optical rotation [α]_{D} (+2)

The second eluted peak was collected and the solvent evaporated to give 5.5 mg of a colourless solid. The absolute stereochemistry was arbitrarily assigned as N-[(3aR,5S,7aS)-7a-(3,4-dimethoxyphenyl)-2-methyloctahydro-1H-isoindol-5-yl]-N′-[4-fluoro-3-(trifluoromethyl)phenyl]urea, HRMS Calc for C₂₅H₂₈F₄N₂O₃: 480.2036, HRMS Found: 408.2053, optical rotation [α]_{D} (−1.5)

### EXAMPLE 54

### N-[4-Bromo-2-(trifluoromethyl)phenyl]-N′-[(3aS*,5R*,7aR*)-7a-(3,4-dimethoxyphenyl)-2-methyloctahydro-1H-isoindol-5-yl]urea trifluoroacetate

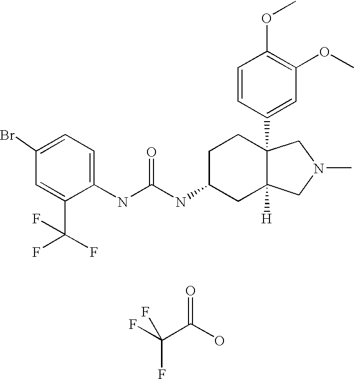

Procedure: as for example 48

Starting material: 4-bromo-2-(trifluoromethyl)phenylisocyanate

Yield: 13%, HPLC purity 96%, m/z 556 (M+H)⁺, HRMS for C₂₅H₂₉BrF₃N₃O₃: calcd: 555.1344, found: 555.1339.

### EXAMPLE 55

### N-(3-Cyanophenyl)-N′-[(3aS*,5R*,7aR*)-7a-(3,4-dimethoxyphenyl)-2-methyloctahydro-1H-isoindol-5-yl]urea trifluoroacetate

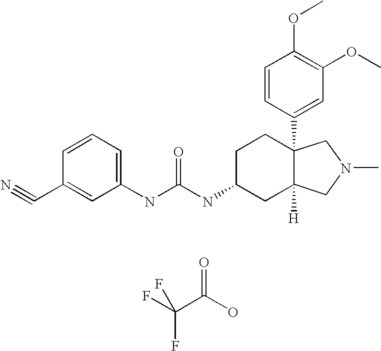

Procedure: as for example 48

Starting material: 3-cyanophenylisocyanate

Yield 22%, HPLC purity 93%, m/z 435 (M+H)⁺, HRMS for C₂₅H₃₀N₄O₃: calcd: 434.2318, found: 434.2302.

### EXAMPLE 56

### N-[(3aS*,5R*,7aR*)-7a-(3,4-Dimethoxyphenyl)-2-methyloctahydro-1H-isoindol-5-yl]-3-(trifluoromethyl)benzamide trifluoroacetate

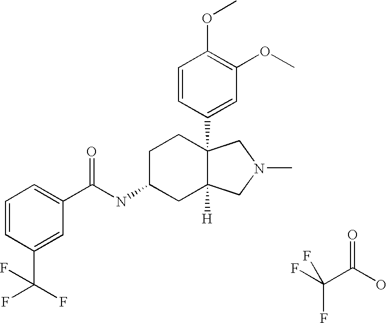

3-(trifluoromethyl)benzoyl chloride (9 mg, 0.0454 mmol) was added a solution of (3aS*,5R*,7aR*)-7a-(3,4-dimethoxyphenyl)-2-methyloctahydro-1H-isoindol-5-amine, prepared in comparative example 6, (12 mg, 0.0413 mmol) in DCM (1 ml) and triethylamine (8 μl, 0.0538 mmol). The mixture was stirred overnight and the solvent evaporated. The product was purified by preparative HPLC to yield 7.1 mg of the title compound. Yield 30%, HPLC purity 93%, m/z 463 (M+H)⁺, HRMS for C₂₅H₂₉F₃N₂O₃: calcd: 462.2130, found: 462.2121.

Preparation of a Pharmaceutical Composition

### EXAMPLE 57

### Preparation of Tablets

| Ingredients | mg/tablet |
|---|---|
| 1. Active compound of formula (I) | 10.0 |
| 2. Cellulose, microcrystalline | 57.0 |
| 3. Calcium hydrogen phosphate | 15.0 |
| 4. Sodium starch glycolate | 5.0 |
| 5. Silicon dioxide, colloidal | 0.25 |
| 6. Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

Biological Methods

The ability of a compound of the invention to bind or act at the MCH1R receptor can be determined using in vitro and in vivo assays known in the art.

Binding Assay

The compounds according to the invention were evaluated for their binding to the human MCH1R receptor by the following method:

Materials and Methods

Materials

Compounds. MCH peptide was purchased from Phoenix pharmaceuticals. (Phe¹³, [¹²⁵I]Tyr¹⁹ Melanine-Concentrating Hormone (human, mouse, rat) ([¹²⁵I]-MCH) was obtained from NEN life Science Products. Inc. Boston, Mass. Wheat germ agglutinine SPA beads (RPNQ 0001) were obtained from Amersham-Pharmacia Biotech. All other reagents used are of highest purity from different resources available. Protein Kits, Micro BCA™ Protein Assay Reagent Kit (Cat No. 23235) were purchased from Piece, Rockford, Ill., USA.

Plastic wares. Cell culture flasks, dishes were from Decton Dickinson Labware, NJ, USA. Scintillation plate, white clear bottom were from Wallac, Finland.

Cells and Culture Conditions

CHO-K1 hMCHRI (Euroscreen, Brussels, Belgium, # ES-370-C) were cultivated in Nutrient mixture Ham's F-12 with Glutamax I (Gibco-BRL #31765-027) supplemented with 10% heat-inactivated foetal calf serum (FCS, Gibco-BRL #10108-165) and 400 μg/ml geniticin (Gibco-BRL #1140-0359). The cells were sub-cultivated twice weekly with split ratio=1:20-1:30. For membrane preparation the cells were cultured in 500 mm² dishes and the cells were harvested when 90% confluent.

Membrane Preparation

When the cells reached more than 90% confluence, dishes (500 cm²) were rinsed twice with 20 ml PBS (Ca²⁺ and Mg²⁺ free). Buffer A, which contains Tris.HCl (15), MgCl₂.6H₂O (2), EDTA (0.3), EGTA (1) in mM with pH 7.5, 25 ml was added and cells were suspended using a window scraper. The cells were collected in 50 ml Falcon tube pre-cooled on ice and then centrifuged for 3 minutes at 1500 g at 4° C. The supernatant was discarded and the cells were suspended again with Buffer A. The cells were homogenized using a Polytron homogenizer at setting 4 for 4 times for 30 seconds with 1 minute pause between the cycles. The homogenized preparation was centrifuged at 40,000 g (18500 rpm with ss-34, No. 5 rotor in Sorvall centrifuge, RCSC, DuPont) for 25 minutes at 4° C. The pellets were washed once with Buffer A and centrifuged again under the same conditions. The pellets were suspended with Buffer B, which contains Tris.HCl (7.5), MgCl₂.6H₂O (12.5), EDTA (0.3), EGTA (1), sucrose (25) in mM with pH 7.5, and gently homogenized for several times with a glass homogenizer. The membrane preparation was aliquoted into Eppendorf tubes, 1 ml/tube and frozen at −70° C.

Membrane Protein Determination

The protein determination was done as described in the instruction provided with Pierce protein assay kit (Peirce Micro BCA Protein assay reagent kit, No 23235, Pierce, USA).

Receptor Binding by SPA

The WGA beads were re-constituted with reaction buffer, which contains Tris (50), MgCl₂ (5), EDTA (2.5) in mM with pH adjusted to 7.4, to 40 mg/ml as a stock suspension. To link the membrane with the bead, the beads and the membrane will be pre-incubated with for 30 minutes at room temperature with gentle shaking. The suspension of the beads was centrifuged at 3400 rpm for 2 minutes using centrifuge. The supernatant was discarded and the beads were re-suspended with binding buffer, HEPES (25 mM), MgCl₂ (5 mM), CaCl₂ (1 mM), BSA (0.5%) with peptidase inhibitors (1 μg/ml) Leupeptin, Aprotinin and pepstatin, pH 7.4.

The radio labeled [¹²⁵I]-MCH was diluted with cold MCH in ratio 1:3. In Kd determination, the concentrations of labeled peptide were 3 nM with 1:2 series dilution for 11 samples. The amount of the beads was 0.25 mg/well. The results were calculated using Excel program and the curves were drawn using a program GraphPad Prism.

For screening of the substances the amount of the beads used was 0.25 mg/well and the amount of the membrane protein was 4 μg/well. In all displacement experiments, 0.2 nM [¹²⁵I]-MCH was used for total binding and 300 nM MCH used as non-specific binding. The total volume was 200 μl, which contained 50 μl [¹²⁵I]-MCH, 100 μl substances and 50 μl beads. The plate was gently shaken for 30 minute and incubated overnight. The samples were counted using Microbeta counter (Wallac Trilux 1450 Micro beta counter, Wallac, Finland) for 2 minutes and the results were calculated by using the computer program Activity Base.

Results

The Kᵢ value is calculated from IC₅₀ using the Cheng Prushoff equation: Kᵢ=IC₅₀(1+[S]/Kₘ) [Cheng, Y. C.; Prushoff, W. H. Biochem. Pharmacol. 1973, 22, 3099-3108].

The compounds of formula (I) exhibit the IC₅₀ values for the MCH1R receptor in the range from 1 nM to 10 μM. Illustrative of the invention, the following Ki values have been determined in the assay (see Table 1):

TABLE 1
Ki values determined in the assay.

| Compound of Example | Ki (nM) |
|---|---|
| 44 | 288 |
| 48 | 74 |

1. A compound of the general formula (I) 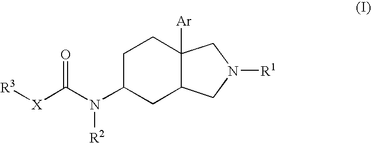
or a pharmaceutically acceptable salt, hydrates, geometrical isomers, racemates, tautomers, optical isomers, N-oxides and prodrug forms thereof, wherein:
X is NH or a single bond;
Ar is aryl optionally, independently substituted by one or more C₁₋₆-alkoxy;
R¹ is C₁₋₆-alkyl;
R² is H;
R³ is selected from aryl optionally, independently substituted by one or more of halogen, cyano, halo-C₁₋₆-alkylthio, C₁₋₆-alkoxy, C₁₋₆-alkyl, halo-C₁₋₆-alkyl; and heteroaryl optionally, independently substituted by one or more halogen.

2. The compound according to claim 1 wherein Ar is phenyl optionally, independently substituted by one or more C₁₋₆-alkoxy.

3. The compound of claim 1 wherein the phenyl is optionally, independently substituted by one or more methoxy.

4. The compound of claim 1 wherein Ar is 3,4-dimethoxyphenyl.

5. The compound of claim 1 wherein R¹ is methyl.

6. The compound of claim 1 wherein R³ is selected from phenyl optionally, independently substituted by one or more of bromo, chloro, cyano, (difluoromethyl)thio, fluoro, iodo, methoxy, methyl, trifluoromethyl, (trifluoromethyl)thio; 1,3-benzodioxol-5-yl optionally substituted by one or more of fluoro; and pyridyl.

7. The compound of claim 1 wherein R³ is selected from 4-bromo-3-methylphenyl, 3-bromophenyl, 4-bromophenyl, 4-bromo-2-(trifluoromethyl)phenyl, 3-chloro-2-fluorophenyl, 3-chloro-4-fluorophenyl, 3-chlorophenyl, 4-chlorophenyl, 4-chloro-3-(trifluoromethyl)phenyl, 3-cyanophenyl, 4-cyanophenyl, 2,4-dichlorophenyl, 2,5-dichlorophenyl, 3,4-dichlorophenyl, 2,2-difluoro-1,3-benzodioxol-5-yl, 3-(difluoromethyl)thiophenyl, 2,5-difluorophenyl, 3,4-difluorophenyl, 3,5-difluorophenyl, 3,4-dimethoxyphenyl, 3,5-dimethylphenyl, 3-fluorophenyl, 4-fluorophenyl, 2-fluoro-3-(trifluoromethyl)phenyl, 2-fluoro-4-(trifluoromethyl)phenyl, 3-fluoro-4-(trifluoromethyl)phenyl, 3-fluoro-5-(trifluoromethyl)phenyl, 4-fluoro-3-(trifluoromethyl)phenyl, 4-iodophenyl, 2,3,4-trifluorophenyl, 3,4,5-trifluorophenyl, 3-(trifluoromethyl)phenyl, 4-(trifluoromethyl)phenyl, 4-(trifluoromethyl)thiophenyl, and 4-pyridyl.

8. The compound of claim 1, selected from the group consisting of:
N-[(3aS*,5R*,7aR*)-7a-(3,4-dimethoxyphenyl)-2-methyloctahydro-1H-isoindol-5-yl]-4-iodobenzamide;
N-[(3aS*,5R*,7aR*)-7a-(3,4-dimethoxyphenyl)-2-methyloctahydro-1H-isoindol-5-yl]-3,4-dimethoxybenzamide trifluoroacetate;
4-chloro-N-[(3aS*,5R*,7aR*)-7a-(3,4-dimethoxyphenyl)-2-methyloctahydro-1H-isoindol-5-yl]benzamide;
N-[(3aS*,5R*,7aR*)-7a-(3,4-dimethoxyphenyl)-2-methyloctahydro-1H-isoindol-5-yl]-4-fluorobenzamide;
4-bromo-N-[(3aS*,5R*,7aR*)-7a-(3,4-dimethoxyphenyl)-2-methyloctahydro-1H-isoindol-5-yl]benzamide;
N-[(3aS*,5R*,7aR*)-7a-(3,4-dimethoxyphenyl)-2-methyloctahydro-1H-isoindol-5-yl]-3-fluoro-4-(trifluoromethyl)benzamide;
4-bromo-N-[(3aS*,5R*,7aR*)-7a-(3,4-dimethoxyphenyl)-2-methyloctahydro-1H-isoindol-5-yl]-3-methylbenzamide;
N-[(3 aS*,5R*,7aR*)-7a-(3,4-dimethoxyphenyl)-2-methyloctahydro-1H-isoindol-5-yl]-4-fluoro-3-(trifluoromethyl)benzamide;
N-[(3aS*,5R*,7aR*)-7a-(3,4-dimethoxyphenyl)-2-methyloctahydro-1H-isoindol-5-yl]-4-(trifluoromethyl)benzamide;
N-[(3aS*,5R*,7aR*)-7a-(3,4-dimethoxyphenyl)-2-methyloctahydro-1H-isoindol-5-yl]-2,2-difluoro-1,3-benzodioxole-5-carboxamide;
N-(3-chloro-4-fluorophenyl)-N′-[(3aS*,5R*,7aR*)-7a-(3,4-dimethoxyphenyl)-2-methyloctahydro-1H-isoindol-5-yl]urea trifluoroacetate;
N-[4-chloro-3-(trifluoromethyl)phenyl]-N′-[(3aS*,5R*,7aR*)-7a-(3,4-dimethoxyphenyl)-2-methyloctahydro-1H-isoindol-5-yl]urea trifluoroacetate;
N-(3,5-difluorophenyl)-N′-[(3aS*,5R*,7aR*)-7a-(3,4-dimethoxyphenyl)-2-methyloctahydro-1H-isoindol-5-yl]urea trifluoroacetate;
N-[(3aS*,5R*,7aR*)-7a-(3,4-dimethoxyphenyl)-2-methyloctahydro-1H-isoindol-5-yl]-N′-(2,3,4-trifluorophenyl)urea trifluoroacetate;
N-[(3aS*,5R*,7aR*)-7a-(3,4-dimethoxyphenyl)-2-methyloctahydro-1H-isoindol-5-yl]-N′-(3-fluorophenyl)urea trifluoroacetate;
N-(3-chlorophenyl)-N′-[(3aS*,5R*,7aR*)-7a-(3,4-dimethoxyphenyl)-2-methyloctahydro-1H-isoindol-5-yl]urea trifluoroacetate;
N-[(3aS*,5R*,7aR*)-7a-(3,4-dimethoxyphenyl)-2-methyloctahydro-1H-isoindol-5-yl]isonicotinamide 1-oxide trifluoroacetate;
N-(3,4-difluorophenyl)-N′-[(3aS*,5R*,7aR*)-7a-(3,4-dimethoxyphenyl)-2-methyloctahydro-1H-isoindol-5-yl]urea;
N-[(3aS*,5R*,7aR*)-7a-(3,4-dimethoxyphenyl)-2-methyloctahydro-1H-isoindol-5-yl]-N′-[3-fluoro-5-(trifluoromethyl)phenyl]urea acetate;
N-[(3aS*,5R*,7aR*)-7a-(3,4-dimethoxyphenyl)-2-methyloctahydro-1H-isoindol-5-yl]-N′-[2-fluoro-3-(trifluoromethyl)phenyl]urea acetate;
N-(2,5-difluorophenyl)-N′-[(3aS,5R,7aR)-7a-(3,4-dimethoxyphenyl)-2-methyloctahydro-1H-isoindol-5-yl]urea;
3,4-dichloro-N-[(3aS*,5R*,7aR*)-7a-(3,4-dimethoxyphenyl)-2-methyloctahydro-1H-isoindol-5-yl]benzamide;
N-[(3aS*,5R*,7aR*)-7a-(3,4-dimethoxyphenyl)-2-methyloctahydro-1H-isoindol-5-yl]-N′-(3,4,5-trifluorophenyl)urea;
N-[(3aS*,5S*,7aR*)-7a-(3,4-dimethoxyphenyl)-2-methyloctahydro-1H-isoindol-5-yl]-N′-[2-fluoro-3-(trifluoromethyl)phenyl]urea acetate;
3,4-dichloro-N-[(3aS*,5S*,7aR*)-7a-(3,4-dimethoxyphenyl)-2-methyloctahydro-1H-isoindol-5-yl]benzamide;
N-[(3 aS*,5R*,7aR*)-7a-(3,4-dimethoxyphenyl)-2-methyloctahydro-1H-isoindol-5-yl]-N′-[4-fluoro-3-(trifluoromethyl)phenyl]urea trifluoroacetate;
N-[(3aS,5R,7aR)-7a-(3,4-dimethoxyphenyl)-2-methyloctahydro-1H-isoindol-5-yl]-N′-[4-fluoro-3-(trifluoromethyl)phenyl]urea;
N-[(3aR,5S,7S)-7a-(3,4-dimethoxyphenyl)-2-methyloctahydro-1H-isoindol-5-yl]-N′-[4-fluoro-3-(trifluoromethyl)phenyl]urea;
N-[(3aS*,5R*,7aR*)-7a-(3,4-dimethoxyphenyl)-2-methyloctahydro-1H-isoindol-5-yl]-3-fluorobenzamide trifluoroacetate;
3-chloro-N-[(3aS*,5R*,7aR*)-7a-(3,4-dimethoxyphenyl)-2-methyloctahydro-1H-isoindol-5-yl]benzamide trifluoroacetate;
3-bromo-N-[(3aS*,5R*,7aR*)-7a-(3,4-dimethoxyphenyl)-2-methyloctahydro-1H-isoindol-5-yl]benzamide trifluoroacetate;
3-[(difluoromethyl)thio]-N-[(3aS*,5R*,7aR*)-7a-(3,4-dimethoxyphenyl)-2-methyloctahydro-1H-isoindol-5-yl]benzamide trifluoroacetate;
3-Cyano-N-[(3aS*,5R*,7aR*)-7a-(3,4-dimethoxyphenyl)-2-methyloctahydro-1H-isoindol-5-yl]benzamide trifluoroacetate;
4-Cyano-N-[(3aS*,5R*,7aR*)-7a-(3,4-dimethoxyphenyl)-2-methyloctahydro-1H-isoindol-5-yl]benzamide trifluoroacetate;
N-[(3aS*,5R*,7aR*)-7a-(3,4-dimethoxyphenyl)-2-methyloctahydro-1H-isoindol-5-yl]-3,4-difluorobenzamide trifluoroacetate;
N-[(3aS*,5R*,7aR*)-7a-(3,4-dimethoxyphenyl)-2-methyloctahydro-1H-isoindol-5-yl]-3-fluoro-5-(trifluoromethyl)benzamide trifluoroacetate;
N-[(3aS*,5R*,7aR*)-7a-(3,4-dimethoxyphenyl)-2-methyloctahydro-1H-isoindol-5-yl]-2-fluoro-4-(trifluoromethyl)benzamide trifluoroacetate;
N-[(3aS*,5R*,7aR*)-7a-(3,4-dimethoxyphenyl)-2-methyloctahydro-1H-isoindol-5-yl]-4-[(trifluoromethyl)thio]benzamide trifluoroacetate;
3-chloro-N-[(3aS*,5R*,7aR*)-7a-(3,4-dimethoxyphenyl)-2-methyloctahydro-1H-isoindol-5-yl]-2-fluorobenzamide trifluoroacetate;
N-[(3aS*,5R*,7aR*)-7a-(3,4-dimethoxyphenyl)-2-methyloctahydro-1H-isoindol-5-yl]-2,5-difluorobenzamide trifluoroacetate;
N-[(3aS*,5R*,7aR*)-7a-(3,4-dimethoxyphenyl)-2-methyloctahydro-1H-isoindol-5-yl]-N′-(3,5-dimethylphenyl)urea trifluoroacetate;
N-(2,5-dichlorophenyl)-N′-[(3aS*,5R*,7aR*)-7a-(3,4-dimethoxyphenyl)-2-methyloctahydro-1H-isoindol-5-yl]urea trifluoroacetate;
N-(2,4-dichlorophenyl)-N′-[(3aS*,5R*,7aR*)-7a-(3,4-dimethoxyphenyl)-2-methyloctahydro-1H-isoindol-5-yl]urea trifluoroacetate;
N-[(3aS*,5R*,7aR*)-7a-(3,4-dimethoxyphenyl)-2-methyloctahydro-1H-isoindol-5-yl]-N′-(4-iodophenyl)urea trifluoroacetate;
N-[(3aS,5R,7aR)-7a-(3,4-dimethoxyphenyl)-2-methyloctahydro-1H-isoindol-5-yl]-3-fluoro-4-(trifluoromethyl)benzamide trifluoroacetate;
N-[(3aR,5S,7aS)-7a-(3,4-dimethoxyphenyl)-2-methyloctahydro-1H-isoindol-5-yl]-3-fluoro-4-(trifluoromethyl)benzamide trifluoroacetate;
N-[4-bromo-2-(trifluoromethyl)phenyl]-N′-[(3aS*,5R*,7aR*)-7a-(3,4-dimethoxyphenyl)-2-methyloctahydro-1H-isoindol-5-yl]urea trifluoroacetate;
N-(3-cyanophenyl)-N′-[(3aS*,5R*,7aR*)-7a-(3,4-dimethoxyphenyl)-2-methyloctahydro-1 H-isoindol-5-yl]urea trifluoroacetate; and
N-[(3aS*,5R*,7aR*)-7a-(3,4-dimethoxyphenyl)-2-methyloctahydro-1H-isoindol-5-yl]-3-(trifluoromethyl)benzamide trifluoroacetate.

9. A process for the preparation of a compound according to claim 1 comprising any of the following steps:
(a) acylation of an amine with a substituted benzoyl chloride,
(b) treatment of an amine with 4-carboxypyridine-N-oxide in the presence of a coupling agent,
(c) treatment of an amine with a substituted aryl isocyanate, or
(d) treatment of an amine with triphosgene and another amine.

10. A pharmaceutical formulation containing a compound according to claim 1 and a pharmaceutically acceptable diluent or carrier.

11. A method for the treatment or prophylaxis of obesity, diabetes mellitus, hyperlipidemia, hyperglycemia, depression, anxiety, urinary incontinence, and for modulation of appetite, said method comprising administering to a subject in need of such treatment an effective amount of a compound according claim 1.

12. A method for the treatment or prophylaxis of disorders related to the MCH1R receptor and for modulation of appetite, said method comprising administering to a subject in need of such treatment an effective amount of a compound according claim 1.

13. A method according to claim 12, wherein the disorders are selected from obesity, diabetes mellitus, hyperlipidemia, hyperglycemia, depression, anxiety, and urinary incontinence.

14. A method for modulating MCH1R receptor activity, comprising administering to a subject in need thereof an effective amount of a compound according to claim 1.

15. The pharmaceutical formulation of claim 10 further comprising a anti-obesity medicine.

16. A method for treating obesity comprising administering a compound according to claim 1 and an anti-obesity medicine.
